(19) United States
(12) Patent Application Publication
(10) Pub. No.: US 2016/0324465 A1
(43) Pub. Date: Nov. 10, 2016

(54) SYSTEMS AND METHODS FOR DIAGNOSING SLEEP

(71) Applicants: Laszlo Osvath, Dundas (CA); Colin Shapiro, Dundas (CA)

(72) Inventors: Laszlo Osvath, Dundas (CA); Colin Shapiro, Dundas (CA)

(21) Appl. No.: 15/110,566

(22) PCT Filed: Jan. 8, 2015
(86) PCT No.: PCT/CA2015/000010

§ 371 Date: Jul. 8, 2016

Related U.S. Application Data
(60) Provisional application No. 61/925,177, filed on Jan. 8, 2014.

(51) Int. Cl. A61B 5/00; A61B 5/0492; A61B 5/18; A61B 5/0478

(52) U.S. Cl. CPC A61B 5/4812; A61B 5/0478; A61B 5/0492; A61B 5/6814; A61B 5/18; A61B 5/4088; A61B 5/4821

(57) ABSTRACT

Systems and methods for sleep stage determination are disclosed. Example systems disclosed herein includes a complexity module operable to measure the complexity of regularities in an EEG channel, and a stager operable to output at least one corresponding sleep stage. Some example systems also include monitoring a subject, and determine the subject may have impairment, Alzheimer's disease, or anesthesia problem that is associated with sleep staging problem.

### RELATED APPLICATIONS

This application claims the benefit of U.S. provisional patent application Ser. No. 61/925,177 filed Jan. 8, 2014, the entire contents of which are hereby incorporated by reference herein.

### TECHNICAL FIELD

The embodiments described herein relate to systems and methods for sleep stage determination, and in particular to systems and methods for sleep stage determination that may be suitable for performance outside of a sleep laboratory.

### INTRODUCTION

Sleep is one of the basic mammalian needs. For example, the state of wakefulness of a person has an effect on sleep states, and the quality of sleep often has a significant impact on daytime (i.e., non-sleep) functioning of a person. Sleep disorders that interfere with sleep quality can have significant individual and societal consequences, including causing issues such as hypertension, cardiovascular disease, obesity and diabetes.

Currently, sleep recording for diagnostic purposes (i.e., to diagnose sleep disorders) is performed in sleep laboratories, and is called polysomnography (PSG).

Polysomnography generally involves the acquisition of a number of different signals of a subject. Three of these groups of signals (namely cerebral activity, skeletal muscle tone, and electrooculogram) can be summarized in a hypnogram, which represents the totality of sleep stages (i.e., levels and types of sleep) that occur during a sleep session.

Determining which “stage” of sleep a subject is experiencing during a sleep session is routinely performed by sleep technologists who manually identify each stage based on standard scoring criteria.

For example, stage 1 is the beginning of a sleep cycle, which is relatively light sleep. During this stage, the brain produces alpha waves. However, during stage 2 sleep, the brain produces rapid, rhythmic brain wave activity known as sleep spindles. In stage 3, which is a transitional stage between light and deep sleep, the brain begins to produce delta waves, which are slow. Then, in stage 4, the brain is in a deep sleep and produces many delta waves (depending on the particular sleep classification system being used, in some cases stage 3 sleep and stage 4 sleep may be grouped together and referred to simply as slow-wave sleep (SWS)). Finally, in stage 5, the brain enters Rapid Eye Movement (REM) sleep, also known as active sleep. This is the stage in which the majority of dreaming will occur.

### BRIEF DESCRIPTION OF THE DRAWINGS

Some embodiments will now be described, by way of example only, with reference to the following drawings, in which:

FIG. 1 is a schematic diagram illustrating a conventional placement of electrodes on a subject's head for a polysomnography (PSG) recording;

FIG. 2 is a schematic diagram illustrating a new placement of electrodes on the head of a subject for a PSG according to the embodiments as described herein;

FIG. 3A is an exemplary graph showing a deep sleep EEG for a subject recorded with a conventional electrode placement, with filter settings set at 1-70 Hz, 60 Hz Notch, 30 s/page, and 7 uV/mm;

FIG. 3B is an exemplary graph showing the same segment of EEG for the subject from FIG. 3A and using same filter settings as in FIG. 3A, but recorded with the electrode placement according to the teachings herein;

FIG. 4 is an exemplary graph showing EEG recorded during REM sleep for a subject with an electrode placement according to the teachings herein, using the same filter settings as in FIG. 3A;

FIG. 5 is a schematic block diagram of a system for determining sleep stages according to one embodiment;

FIG. 6 is a graph showing a frequency characteristic of a Low-Pass filter for use with the system of FIG. 5 according to one embodiment;

FIG. 7 is a graph showing a frequency characteristic of a High-Pass filter for use with the system of FIG. 5 according to one embodiment;

FIG. 8 is a graph showing a frequency characteristic of a Notch filter for use with the system of FIG. 5 according to one embodiment;

FIG. 9 is a schematic block diagram of a REM/SEN density estimator for the system of FIG. 5 according to some embodiments;

FIG. 10 is an exemplary graph showing REM activity on EOG channels (LOC, ROC) according to one embodiment;

FIG. 11 is a schematic block diagram of a stager for use with the system of FIG. 5 according to one embodiment;

FIG. 12A is an exemplary graph of a sleep stage determination for a subject as made manually by a human reviewer using standard scoring criteria;

FIG. 12B is an exemplary graph of an automated sleep stage determination made for the same subject as in FIG. 12A, and showing the complexity of EEG during a sleep session (normalized complexity vs. time). The top horizontal line represents the boundary of N1 and the bottom line represents the top boundary of N2.

FIG. 13 is an exemplary graph showing the border between W-S1 as the highest local minimum before sleep onset (at point X), with the graph representing normalized complexity vs. time;

FIG. 14 is an exemplary graph of a transition W-S1-S2 for alpha generator in a subject (shown as dominant frequency vs. time);

FIG. 15 is an exemplary graph of a beta DPA for a whole session of sleep (shown as percent beta vs. time). The top bar and bottom bar represent the tails of the beta distribution.

FIG. 16 is an exemplary graph, with the top portion of the graph showing normalized complexity, while the bottom portion of the graph shows a first derivative of complexity (in black) and a second derivative of complexity (in grey), with the point A representing the S1-S2 boundary;

FIG. 17 is an exemplary histogram of the error in determination of sleep onset according to one embodiment. On the abscissa the numbers represent epochs (30 s).

FIG. 18 is an exemplary histogram of the error in determination of the REM latency according to one embodiment. On the abscissa the numbers represent epochs (30 s).

FIG. 19 is an exemplary histogram of the error in determination of the DS onset according to one embodiment. On the abscissa the numbers represent epochs (30 s).

FIG. 20 is an exemplary histogram of the error in determination of the sleep efficiency according to one embodiment. On the abscissa the numbers represent percent error.

FIG. 21 is an exemplary histogram of the error in determination of the Total Deep Sleep according to one embodiment. On the abscissa the numbers represent percent error.

FIG. 22 is an exemplary histogram of the error in determination of the Total Light Sleep (S1+S2) according to one embodiment. On the abscissa the numbers represent percent error.

FIG. 23 is an exemplary histogram of the error in determination of the Total Non-REM according to one embodiment. On the abscissa the numbers represent percent error.

FIG. 24 is an exemplary histogram of the error in determination of the Total REM according to one embodiment. On the abscissa the numbers represent percent error.

FIG. 25 is an exemplary histogram of the error in determination of the Total Sleep Time according to one embodiment. On the abscissa the numbers represent percent error.

FIG. 26 is an exemplary histogram of the error in determination of the Total time in stage Wake after sleep onset according to one embodiment. On the abscissa the numbers represent percent error.

FIG. 27 is a schematic relational diagram in the CDP model according to one embodiment.

### DESCRIPTION OF SOME PARTICULAR EMBODIMENTS

For simplicity and clarity of illustration, where considered appropriate, reference numerals may be repeated among the figures to indicate corresponding or analogous elements or steps. In addition, numerous specific details are set forth in order to provide a thorough understanding of the exemplary embodiments described herein. However, it will be understood by those of ordinary skill in the art that the embodiments described herein may be practiced without these specific details. In other instances, well-known methods, procedures and components have not been described in detail so as not to obscure the embodiments generally described herein.

Furthermore, this description is not to be considered as limiting the scope of the embodiments described herein in any way, but rather as merely describing the implementation of various embodiments.

In some cases, the embodiments of the systems and methods described herein may be implemented in hardware, in software, or a combination of hardware and software. For example, some embodiments may be implemented in one or more computer programs executing on one or more programmable computing devices that include at least one processor, a data storage device (including in some cases volatile and non-volatile memory and/or data storage elements), at least one input device, and at least one output device.

In some embodiments, a program may be implemented in a high level procedural or object-oriented programming and/or scripting language to communicate with a computer system. However, the programs can be implemented in assembly or machine language, if desired. In any case, the language may be a compiled or interpreted language.

In some embodiments, the systems and methods as described herein may also be implemented as a non-transitory computer-readable storage medium configured with a computer program, wherein the storage medium so configured causes a computer to operate in a specific and predefined manner to perform at least some of the functions as described herein.

As discussed above sleep recording for diagnostic purposes is currently performed in sleep laboratories. Unfortunately, the setup process involved in determining sleep stages in a sleep laboratory is time consuming.

For instance, conducting a sleep stage investigation requires the placement of a number of electrodes on a subject's head. This electrode placement requires preparation of the recording site for optimal electrical contact. Moreover, according to existing techniques, the placement of electrodes on the subject has to be precise and follow a standardized system (called a 10-20 system), according to which sleep technologists have to measure and identify specific locations on the scalp upon which to place the electrodes.

Some sleep laboratories may use automated software tools to generate hypnograms. However, while these tools have a reasonable degree of accuracy, they are highly dependent on electrode position. This tends to limit their use in certain applications, and prevents the implementation of home studies of sleep stages due. In particular, patients are normally unable to prepare the electrode application sites and place the electrodes with sufficient precision, either by themselves or with the assistance of unskilled personnel, to achieve accurate results.

Furthermore, known software tools have not been generally been tested on the pediatric population (i.e., children) in which the electroencephalography (EEG) readings are different to adult EEG readings.

One of the greatest challenges of modern sleep medicine appears to be its cost effective expansion. Despite the existence of a large number of sleep problems within the population, only a small number of patients are actually ever treated because most conditions pass undetected in normal family medicine practices.

Currently, two of the main barriers that obstruct the process of detection and diagnosis of sleep disorders are educational barriers and technological barriers. The teachings herein are generally addressed at the technological barriers.

Conventional known sleep tests are costly and must be performed in sleep laboratories, which have limited capacity. However, since most of the population does not regularly visit a sleep lab, large numbers of patients remain outside the reach of these laboratories. This has significant public health consequences, for example with respect to issues such as hypertension, cardiovascular disease, obesity and diabetes.

In general, the teachings herein are directed at new systems and methods for human sleep stage determination that are suitable for performance outside of the traditional sleep laboratory setting.

In particular, one of more of the techniques as discussed herein may have one or more benefits over conventional sleep diagnosis techniques, including potential for improved accuracy, greater ease of use, facilitating the possibility of patient self-testing, providing for low-cost diagnosis of sleep disorders, providing sleep stage determination that may be conduced outside of a sleep laboratory, allowing sleep stage determination to be done in patient's home, and providing comparable levels of information as to the levels of information obtained in a conventional in-lab sleep test.

In some cases, the teachings herein may permit the migration of at least a part of some sleep diagnostics away from the sleep laboratories and towards a family medicine-type practice. This may allow for wider scale testing for sleep disorders.

Moreover, in patients where a family medical practitioner (i.e., a doctor or nurse practitioner) detects sleep problems using the teachings herein, patients might then be referred for further specialized diagnostic and treatment in a sleep laboratory. This may make better use of limited healthcare resources, as sleep laboratories could focus more on patients that have already been pre-screened for sleep disorders, and less on patients who may not have any sleep disorders.

Some of the teachings herein may allow a health care practitioner to perform comprehensive sleep tests without a detailed knowledge of sleep medicine, much in the same manner that a family practitioner can currently test blood pressure or temperature.

Furthermore, in some cases the teachings herein might be combined with other mental health, respiratory and/or cardiac diagnostic modules, such as one or more of the modules as described in U.S. provisional patent application Ser. No. 61/828,162 filed May 28, 2013 and entitled “Systems and Methods for Diagnosis of Depression”, the entire contents of which are hereby incorporated by reference herein. Combining the teachings herein with other mental health, respiratory and/or cardiac diagnostic modules may provide for the possibility of highly advanced home diagnostic of sleep, respiration and/or mental disorders.

In some cases, the teachings herein could be used to in the creation of centralized diagnostic hubs, similar to radiology or hematology labs that diagnose a number of comorbid conditions (for instance, in some cases mental disorders, sleep disorders, respiratory and cardiac problems could be diagnosed) that were hitherto diagnosed and treated separately with generally suboptimal outcomes.

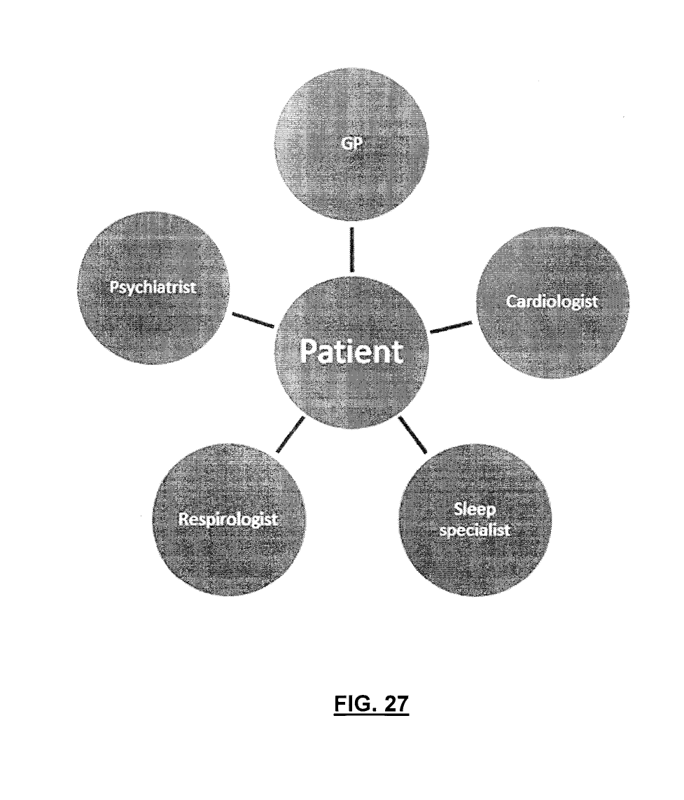

For example, one model of operating central diagnostic points (CDP) for mental health is shown in FIG. 27, where sleep medicine, respirology and cardiology can be performed using automated remote diagnostic technology implemented in the patient's home. A number of physicians from a number of specialties (family practice, psychiatry, sleep medicine, respirology and cardiology) could be affiliated with a central diagnostic point that can service a city, part of a city or a larger geographic area depending on its capacity. The diagnostic point would receive referrals from any physician in the group and would send devices to the patients. The patient will perform home tests for a number of conditions and return the device in person, by mail, or some other means. Alternately the CDP may have its own courier service. The significant advantage stems from detecting comorbid conditions and better care along with large savings for healthcare systems. This may include, for example, detecting along with respiratory, cardiac and sleep problems comorbid mental health problems and treat the patient for all conditions with potentially improved outcomes.

Some of the embodiments described herein may provide at least one significant advantage in that some patients may not have to go to a sleep lab for diagnosis, but can be tested in their homes. One or more diagnostic hubs could then distribute the results of these home tests to one or more physicians or other medical personnel depending on the requisition and any conditions flagged during the home test (and following an appropriate assessment).

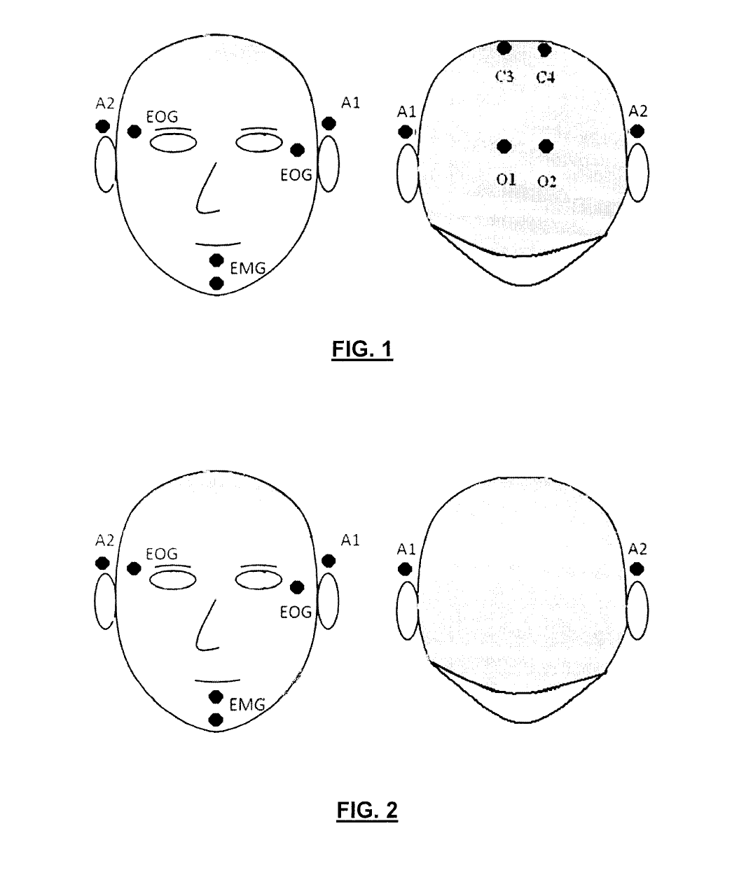

Turning now to the figures, further details of some embodiments will now be described. In particular, FIG. 1 shows a conventional pattern of electrode placement on the head of a patient that is normally used in an in-lab sleep diagnosis.

In contrast, FIG. 2 presents a new pattern of electrode placement according to the teachings herein that may be particularly suitable for use outside of a sleep lab. In particular, this new pattern is designed with a view towards simplifying recording and to permit the application of the electrodes by the patient himself or herself, or in some cases with the assistance of unskilled personnel.

As shown in FIG. 1, in a conventional electrode pattern, scalp electrodes O1, O2, C3, C4 are placed on rearward areas of the patient's scalp that are normally covered with hair.

According to the new pattern of electrode placement shown in FIG. 2, however, these scalp electrodes O1, O2, C3, C4 have been eliminated.

Moreover, the pattern of electrode placement shown in FIG. 2 generally uses a monopolar approach. This approach combines the EEG with a standard electrooculogram and with skeletal muscle activity collected from the temporalis, the submentalis electromyogram (EMG), or both.

One of the unique features of this approach is the collection of EEG from the channels A1-REF, and A2-REF. This arrangement may provide one or more benefits, such as: signals may be directly comparable for artifact rejection; better preservation of spectral purity of signals collected mainly due to lack of interference of contralateral channels that have in general the same frequency content; minimal contamination by the electrical dipole of the eyes (due to greater distance from the source); better separation of sources permitted; signal amplitudes are generally not compromised; all graphoelements are generally present; ease of application; and optionally permitted self-application (i.e. by the patient).

One disadvantage of a low Common Mode Rejection Ratio (CMRR) may be eliminated by internally including a bipolar A1-A2 channel for artifact rejection. Notably, it has been observed that this has only presented importance in less that around 1% of studies.

Table A below presents a brief summary of one montage used for sleep staging according to the teachings herein:

TABLE A
Montage used for sleep staging

A1-REF
A2-REF
LOC-REF
ROC-REF
CHIN1-CHIN2

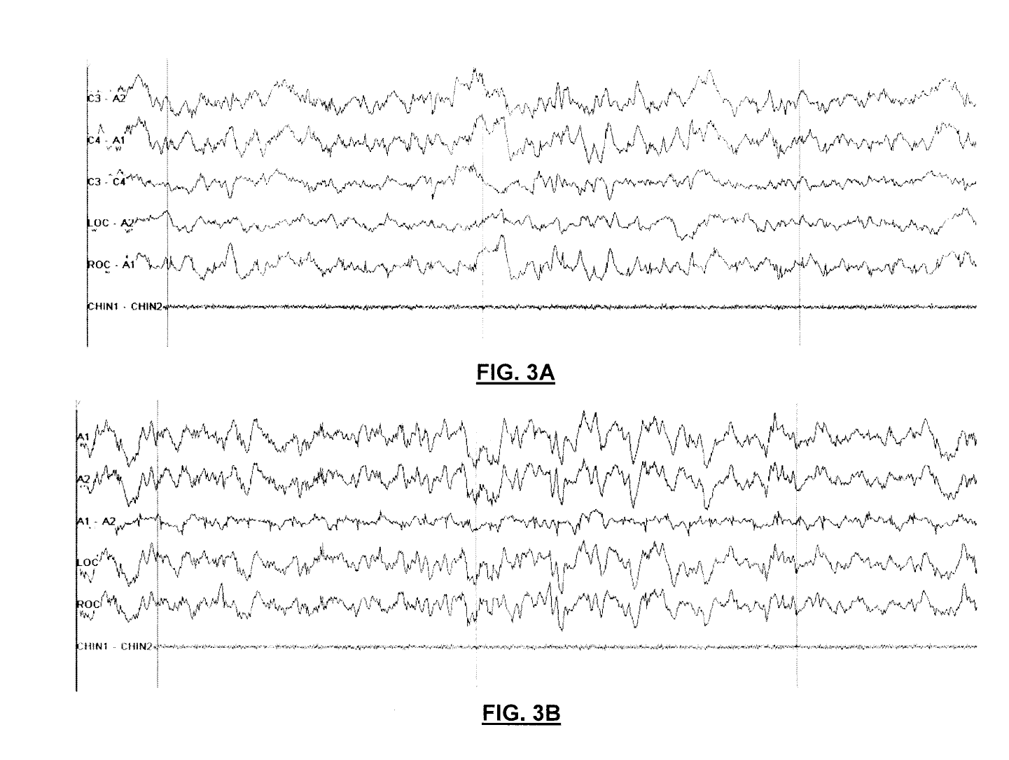

Turning now to FIGS. 3A and 3B, illustrated therein is a comparison of the similarity of amplitude statistics collected using a conventional electrode placement (shown in FIG. 3A) and the new electrode placement described herein (shown in FIG. 3B). In particular, these figure illustrate the similarity of amplitude statistics of delta waves on C3-A2 (FIG. 3A) when compared with A1-REF (FIG. 3B), and between C4-A1 (FIG. 3A) when compared A2-REF (FIG. 3B). In general, this level of agreement is not necessary to practice the teachings herein; however, it can be helpful for the visual validation of the results.

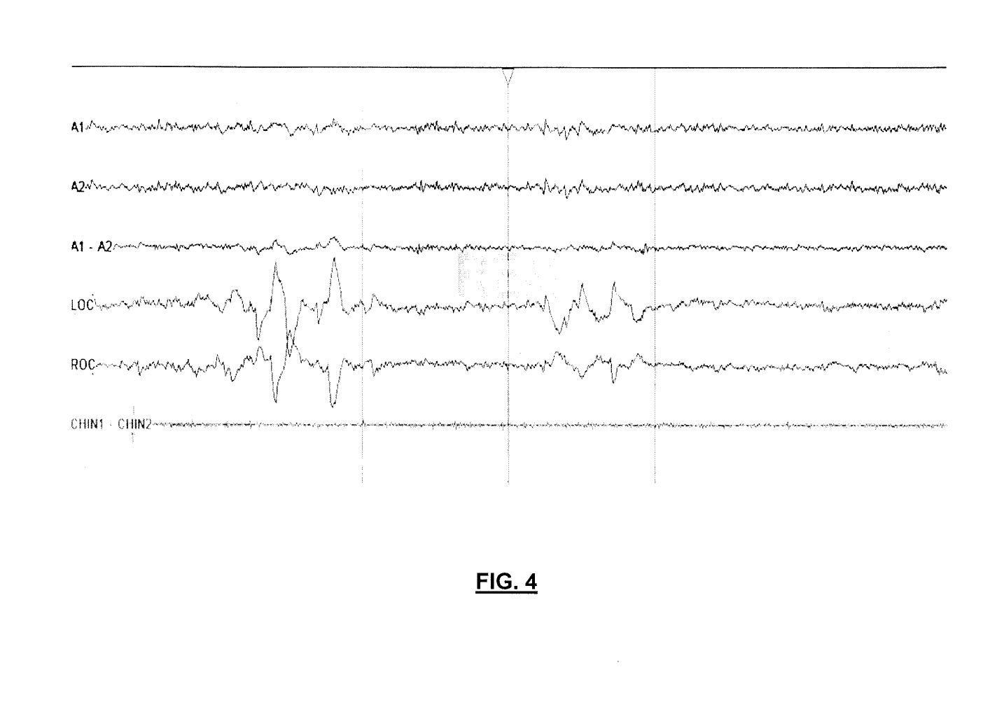

Turning now to FIG. 4, it is apparent by visual inspection that rapid eye movements (REMs) do not contaminate the EEG on the A1 and A2 channels. While this can happen occasionally, the new electrode pattern generally permits better source separation than a bipolar montage, and thus will tend to cause less or even no misinterpretation of the EEG.

In addition to advantages given by the signal quality using this technique, another advantage comes from the ease of application of the electrodes. In particular, the pattern of electrodes shown in FIG. 2 permits a relatively fast self-application of electrodes by a patient or other unskilled personnel without generally compromising diagnostic accuracy.

To provide a better understanding of the teachings herein, a suggestive analogy will now be provided. Sleep can be imagined as a hilly landscape characterized by elevations and landmarks. The sleep landscape is determined by the chronobiological factors. The landmarks are asynchronous, unpredictable events caused by exogenous stimuli interacting with the internal state. Examples of such events can be arousals, awakenings, K complexes, sleep spindles, V waves, and so on. Note that these events are not always present, or visible, and in general do not change the landscape of sleep; they merely decorate the landscape and are conditioned by it.

The teachings as described herein for determining sleep stages and for building a hypnogram can be analogized to directly describing the landscape of sleep.

In contrast, the conventional approach to determining sleep states is more akin to charting the landscape by looking at the flora (i.e., plants and trees) that grow only at specific altitudes of the landscape, and then using this floral information to indirectly figure out the elevation of the landscape.

Following the same analogy, the teachings herein can be used to determine the elevation from direct measurement, while at times the direct measurement may be corroborated with the flora (i.e., plants and trees) that can be found along the way to confirm accuracy of the direct measurement.

As described herein, is has been discovered that this “landscape” of sleep can be determined directly with or without the presence of other “floral” landmarks. One possible advantage of this method is being able to determine the sleep landscape in conditions where the “plants” may not be present (for whatever reasons, which in sleep diagnosis can be due to pathological conditions or controversial cases).

For example, in the real world there are a large number of patients who do not present spindles, alpha activity, or other events. Therefore, the conventional approach to sleep staging for these patients is complicated by the occasional absence of these “floral” elements. These variable conditions can also account for the lack of agreement between different human scorers manually performing a sleep stage determination for the same patient.

We discovered that direct staging of sleep is possible by using the fundamental observation that the complexity of brain processes decreases with the deepening of sleep. Therefore, complexity of brain processes can be used as a direct measure of the depth of sleep.

It has been noted that REM sleep is a state that (in general), presents the highest complexity among sleep states, indicating that the highest level of brain activity occurs during REM sleep. REM sleep is a plateau of consciousness as opposed to all other stages of sleep, and REM sleep is very shallow as compared to other sleep states. One possible explanation can be attributed to the high level of activation of the brain, but saturation of motor neurons, lack of motor activity and muscle tone. This reduces the noise (EMG) superimposed on the EEG.

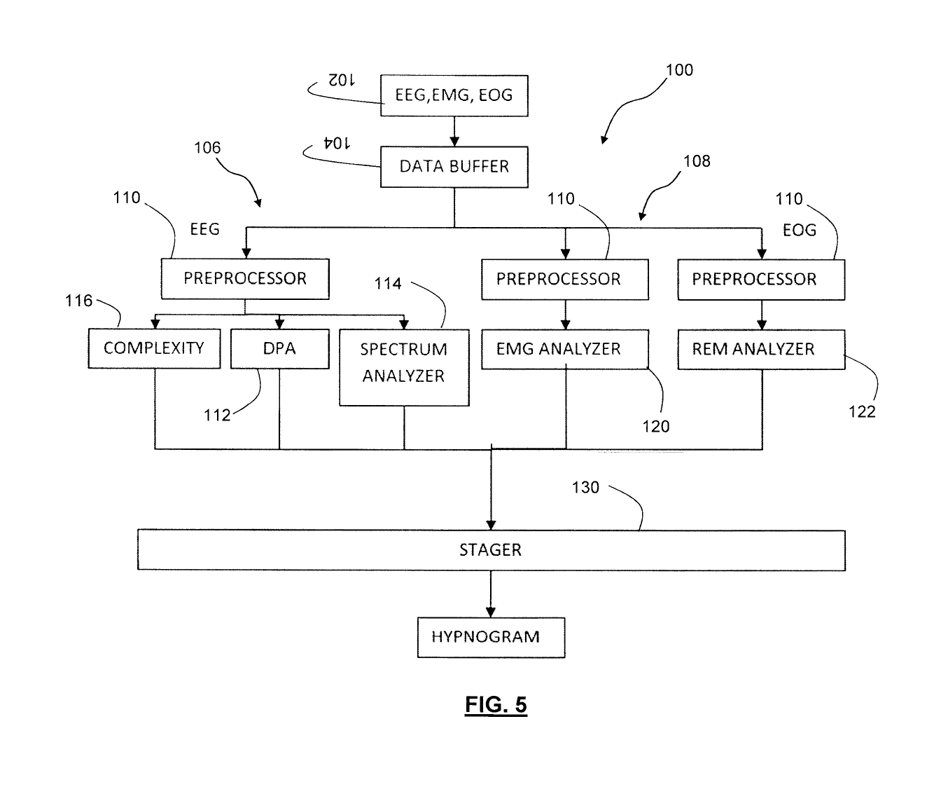

Turning now to FIG. 5, illustrated therein is a schematic block diagram of a system 100 for determining sleep stages according to one embodiment. The system 100 generally includes operational blocks that are functionally adapted to particular processing tasks.

In general, the input 102 to the system 100 is a stream of data packets of variable size, and which may be stored in a buffer 104. In this example, the system 100 generally does the analysis on an epoch-by-epoch basis for each relevant signal type (EEG, EMG, EOG).

In some cases, each signal is extracted channel-by-channel from the data packet. Each channel is then processed specifically for the type of signal that it carries.

Generally, the EEG channel 106 is the main input for the generation of the hypnogram, while the other channels 108 are auxiliary channels, whose role is generally to improve the accuracy of the hypnogram. The following subsections provide further details on the modules of the system 100.

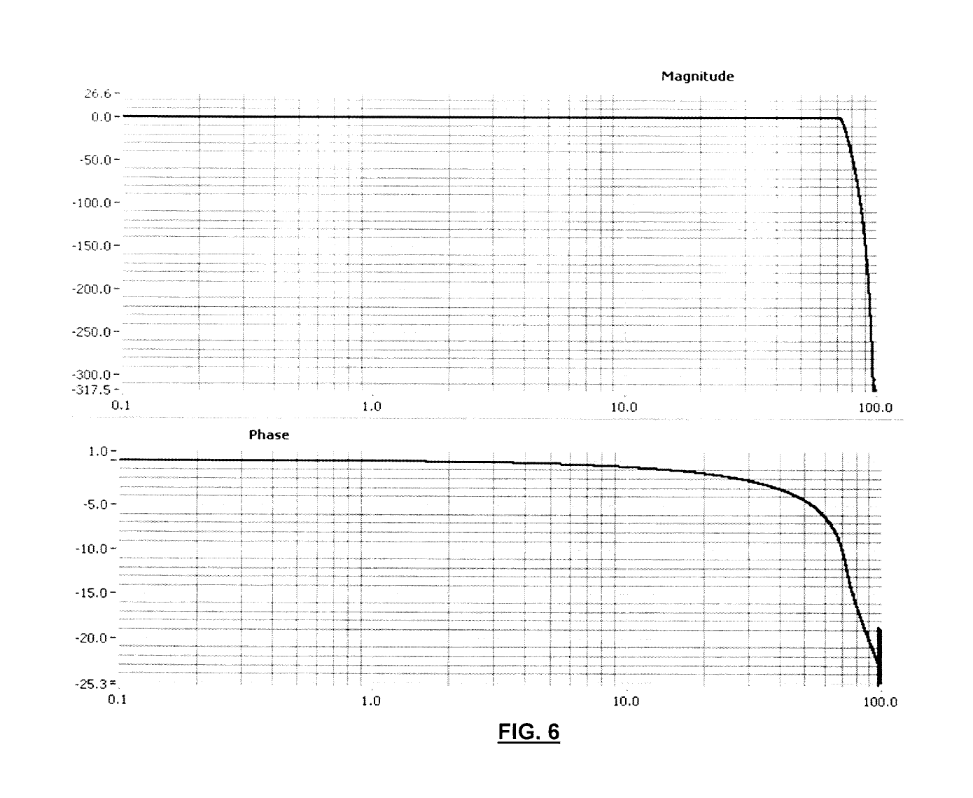

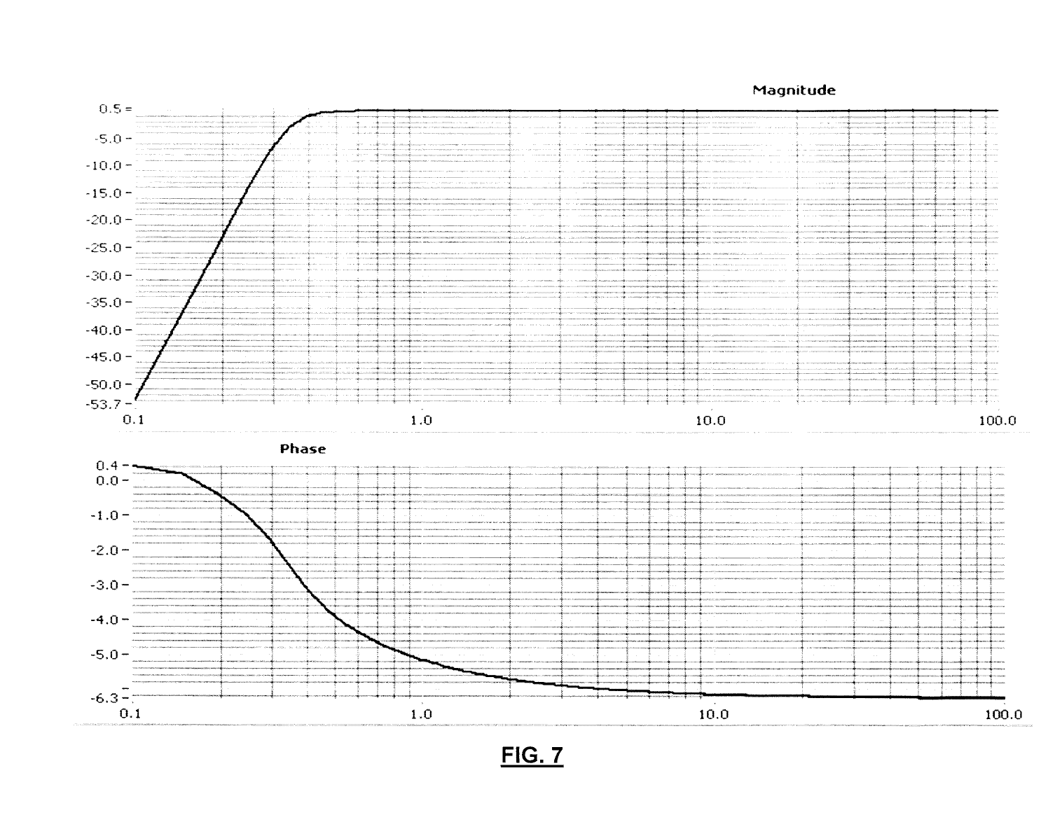

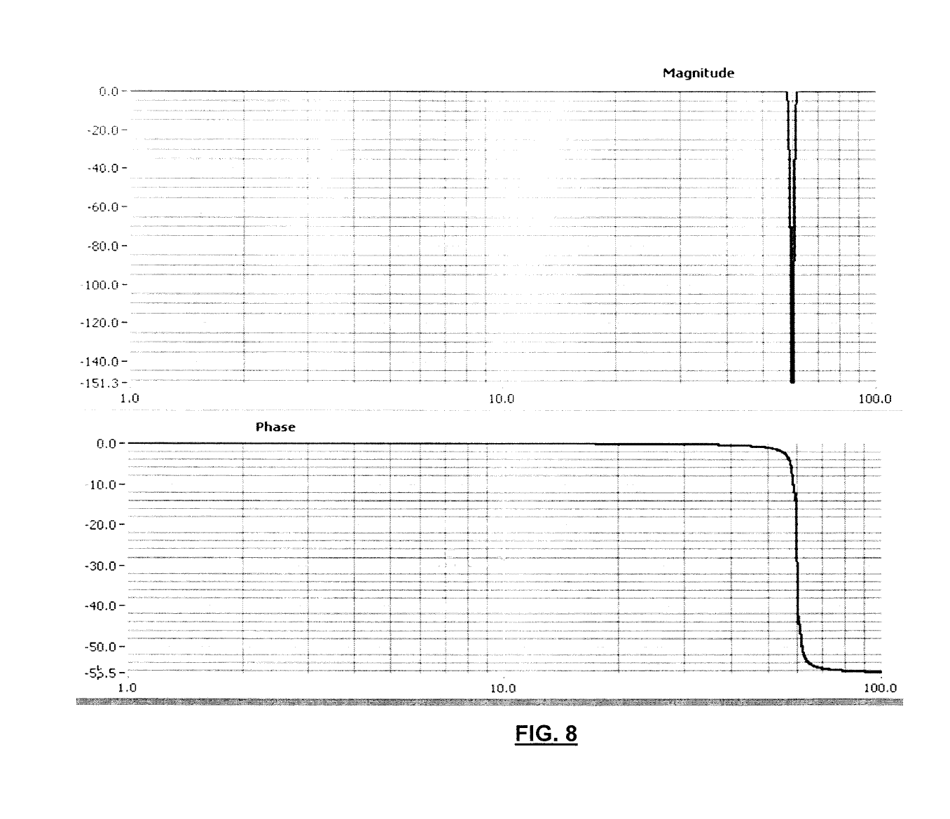

The system 100 includes one or more pre-processor(s) 110. Each pre-processor 110 can apply specific filtering steps to the data depending on the type of input 102. In some cases, filtering may be performed by filters as shown in FIGS. 6-8. For instance, the filtering may be done using digital Butterworth, low-pass and high-pass IIR filters, with −40 dB/dec and corner frequencies at 70 Hz and 0.5 Hz respectively. In some cases, a notch filter and a resampling filter may also be used for cases where the sampling rate is higher than some threshold (i.e., greater than 200 Hz).

The system 100 also includes a Digital Period Analysis (DPA) module 112. In the conventional practice of sleep medicine, the analysis of sleep studies is usually performed in steps of 30 seconds (called epochs). As part of conventional methods of sleep staging, some stages are identified by using proportions of waves of a specified duration and amplitude. Instead of using continuous proportions, a fixed threshold is normally applied, and the epoch is either sub-threshold or above threshold (stages 3 or 4 sleep, for example, are determined based on the density of specific delta waves) depending on the threshold.

The proportions of specific types of waves are informative of certain characteristics of sleep. Using proportions can be considered a more accurate alternative for characterizing sleep than the method of power spectral analysis.

However, the teachings herein are directed at providing an accurate measure of proportion for waves of different durations, i.e. a flow of spectral distribution of waves. For these purposes, the method of counting waves tends to be more adequate than the averaging method of power spectral analysis, because of the closer time-frequency relationship between spectral content and the original time-series.

In particular, according to this technique a specific wave has duration and a corresponding frequency, therefore it is considered either in one band or another band, and the sum of the duration of the waves is always equal to the duration of the original time-series. Variations of this method are known under the name Digital Period Analysis (DPA).

The following text will describe an exemplary version of Digital Period Analysis (DPA). Variations of DPA exist based on the filtering applied prior to segmentation and the segmentation method, however all have the goal of identifying, in a simple way and as well as possible, the wavelet boundaries.

In one specific example, the sample was filtered of the random processes with a digital band-pass Infinite Impulse Response (IIR) filter with −50 db/dec and pass-band (0.5 Hz, 70 Hz). In addition, a digital band-stop filter was for the line frequency. The band stop filter was created using a High-Pass filter with transition band (0.1, 0.5 Hz) with −40 db/dec and a Low-Pass filter with transition-band (70, 80 Hz) −40 db/dec. The characteristics of these filters can be seen in FIGS. 6-8.

The filtering operation transformed the data in a zero mean random variable. The original data will be denoted on the two channels of interest x₁ and x₂ respectively. Each channel will carry a four-dimensional sample of the random process. A section through the process at discrete time n (epoch), will be represented by the random vector:

x[n]=[n_{δ} n_{θ} n_{β}]′ (1)

The resolution in time of the sections is 30 seconds. The significance of the random components will become clear as the computation is undertaken. In particular, the computation of nᵢ where i ∈ {δ, θ, β} proceeds as follows.

An operator that finds the zero crossings of a time series can be defined as:

zx=Zero(x)={n|x|n−1]*x[n]≦0}; x is a random variable

The derivative operator D is then defined:

Dx=x[n]−x[n−1]

Using the operators D and Z, one can build the following random processes:

$${n}_{δ} = {Σ}_{i} ( zx [ i ] - zx [ i - 1 ] \geq \frac{{f}_{s}}{4} ) ( zx [ i ] - zx [ i - 1 ] \leq {f}_{s} ) \qquad ( 1 a )$$

n_{δ} represents the number of waves that have a frequency in the [1, 4 Hz] range. One can then build the set:

zdₓ=Zero(Dx),

and then define the following two random processes:

$$\begin{matrix}{n}_{θ} = {Σ}_{i} ( {zd}_{x} [ i ] - {zd}_{x} [ i - 1 ] \geq \frac{{f}_{s}}{7} ) ( {zd}_{x} [ i ] - {zd}_{x} [ i - 1 ] < \frac{{f}_{s}}{4} ) & ( 1 b ) \\ {n}_{β} = {Σ}_{i} ( {zd}_{x} [ i ] - {zd}_{x} [ i - 1 ] \geq \frac{{f}_{s}}{32} ) * ( {zd}_{x} [ i ] - {zd}_{x} [ i - 1 ] < \frac{{f}_{s}}{16} ) & ( 1 c )\end{matrix}$$

The system 100 also includes a spectrum analyzer 114. For the detection of artifacts and short-lived transients, a higher resolution is generally required than the epoch (30 s). In some cases, a resolution of 3 s is used for spectral analysis. This provides a spectral resolution of 0.3 Hz. This approach is adapted from a multitude of spectral estimation techniques according to the Blackman-Tuckey method:

G_{xy}(θ)=∫_{-π}^{π}N⁻¹|X(θ−λ)|²W(λ)d λ (2)

where W is odd-length symmetric window, N is the width of the window, X is the power spectral density of the process x. Equation (2) is generally easier to compute in time domain:

$${G}_{xx} = {\sum }_{- M}^{M} {k}_{xx} [ n ] w [ n ] {e}^{- i θ n} \\ with \\ {k}_{xx} [ m ] = \frac{1}{N} {\sum }_{0}^{N - 1 - \vert n \vert} x [ i ] x [ i + \vert n \vert ] \qquad ( 3 )$$

A further simplification arises due to the relation between convolution and cross-covariance:

k_{xy}=x*[−n]*y[n] and similarly (4)

kₓₓ=x*[−n]*x[n]

In equation (4), x* is the complex conjugate of x.

Using (4) in (3), one gets the computational relations:

Gₓₓ(θ)=|DFT((x*[−n]*x[n]) w[n])|

One can then compute the dominant frequency for each window (n):

f_{Ld}[n]=argmax(G_{LL}(θ))|_{θ∈[2,30]Hz} (5)

f_{Rd}[n]=argmax(G_{RR}(θ))|_{θ∈[2,30]Hz} (6)

And one can then compute the EMG power:

emgL[n]=Σ₃₅^{notch-5} G_{LL}(θ)+Σ_{notch+5}⁷⁰ G_{LL}(θ) (7)

emgR[n]=Σ₃₅^{notch-5} G_{RR}(θ)+Σ_{notch+5}⁷⁰ G_{RR}(θ) (8)

One can then compute the power in the spindle band:

SpL[n]=Σ_{11.5}^{14.5} G_{LL}(θ) (9)

SpR[n]=Σ_{11.5}^{14.5} G_{RR}(θ) (10)

The system 100 also includes a complexity module 116. Using the “landscape” analogy described above, the complexity module 116 directly determines the landscape of sleep, while the other modules find specific landmarks.

Sleep can be perceived as a reversible alteration of waking consciousness. As the brain descends to deeper states of sleep, the arousability of the brain decreases. Generally, the neural function of the brain during sleep is decreased as compared to wakefulness (although this not true in REM sleep).

At the same time, states of wakefulness and REM sleep are characterized by a lack of additive synchrony, manifested in EEG desynchronization. As the arousability is decreased, the brain “quietens down”. It will be shown herein that measuring the complexity of the neural activity will lead to determining sleep stages.

One immediate problem to be dealt with is how to characterize the complexity of brain processes. Complexity in science is measured in a number of different ways. Entropy is one possible measure, but it has the problem that while the minimum entropy is reflective of synchronized states and low complexity the maximum entropy is reached for states of absolute randomness, which (despite their complex appearance) are actually not equivalent to complexity. In particular, randomness is not equivalent to complexity.

For example, the information about building a human body (i.e., DNA) is encoded in our genes. A random pattern of nucleotide bases will probably result in nothing functional or viable, whereas some specific degree of ordering will create different forms of life. This gives an indication that complexity lies somewhere between order and total disorder.

Another way of characterizing complexity is by finding the shortest code that can describe the object accurately. If one has a redundant sequence TIC TOC TIC TOC TIC TOC TIC TOC, this could be easily characterized by the pseudo code: “repeat TIC TOC 4 times”. A more complicated sequence would require a more complex pseudo code.

The EEG can be considered as a sum of brain activity and noise. The noise carries no information about the state of the brain and ideally our measure of complexity should ignore the noise. Thus, the effective complexity would measure the complexity of the regularities in the EEG and ignore the noise part. This is possible in cases where the noise is small relative to the signal, or if it is possible to remove or separate the noise to work with the signal alone (or both).

The next problem is how to find regularities in the EEG. To this end, the noise can be considered to be small or statistically irrelevant. To address this, a method similar to the Lempel-Ziv approach of data compression was used.

In particular, for each epoch the minimum descriptor length was found that permits one to regenerate the full data (lossless compression). In order to do that, one must build the random variables zₓ,tₓ.

An operator is then defined that finds the ordered set of zero crossings of a time series:

zₓ=Zero(x)

tₓ={zₓ[n]−zₓ[n−1]}

And build a dictionary of durations tₓ with a resolution of 5 ms. We build a set of durations:

T={fs⁻¹, 2*fs⁻¹, 3*fs⁻¹ . . . 256*fs⁻¹}

A wavelet with a duration of 1 second will correspond to the element with value 1;

At each step, an element of the tₓ sequence is coded by emitting binary codes associated with the elements of T, that match elements of tₓ and add to the set T extended sequences of elements of tₓ (two elements, three elements . . . ) that are longer by one as compared to what we already have in T.

This process continues until the set T cannot grow further and we have a full set T. At each step we encode the data using a number of bits N that depends on the cardinality of T at that particular step:

2^{N(t)}>=card(T(t))

The length of the code is dependent on the redundancy of the encoded elements. A regular pattern will be encoded more efficiently and therefore result in shorter code. By measuring the size of the code for the same amount of data (one epoch=30 s), one can get information about the complexity of the data and of the brain function.

The elements of tₓ are replaced by binary codes that encode the longest sequences. The complexity module has a central role in the hypnogram generation.

In some embodiments, there may be another dimension that can be exploited. In particular, it may be possible to execute a dual complexity analysis using both amplitude and time domain. The description outlined above refers to the time domain complexity. The amplitude complexity would be scaling the amplitudes to be in the range [0-255] and applying the same procedure to estimate the amplitude complexity. This way one can obtain another measure of complexity that may be adding some extra information, and which could be helpful in certain situations. However, this additional dimension may further complicate the analysis, and is not necessary.

The system 100 also includes an EMG analyzer 120. The EMG analyzer 120 evaluates skeletal EMG, mainly to assist in separating the REM state. A separate EMG estimation may be performed on the Temporalis muscle in the Spectrum Analyzer module 114.

In particular, the EMG tone may be estimated with a resolution of 3 seconds. We then build the set of zero derivatives of the EMG signal:

Zx=Zero(D emg),

where we applied to the EMG signal (emg) the derivative operators (D and Zero) defined in the DPA section.

EMG_CHIN[n][k]=median({emg[Zx[i]−emg[Zx[i−1]|Zx[i]>3*k*fs, Zx[i]<3*(k+1)*fs], k<eplen/3})

The estimated value of EMG for epoch n and segment k is the median of the segments delimited by the zeros of the first derivative of the signal.

The system 100 also includes a REM/SEM detector 122. Prior to entering the REM/SEM detector 122, the data may be filtered with a band-pass filter, for example a filter with pass band boundaries (0.5, 10 Hz) and a notch filter in a preprocessor 110 (as described above).

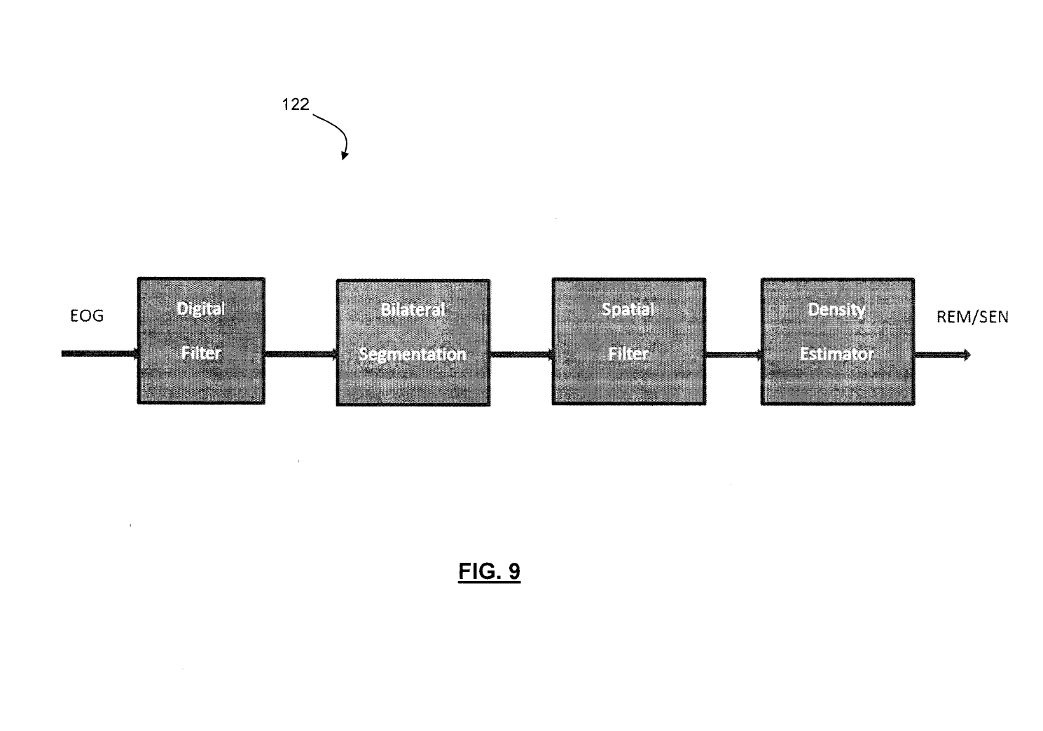

A block diagram of an exemplary REM/SEN density estimator 122 is shown in greater detail in FIG. 9.

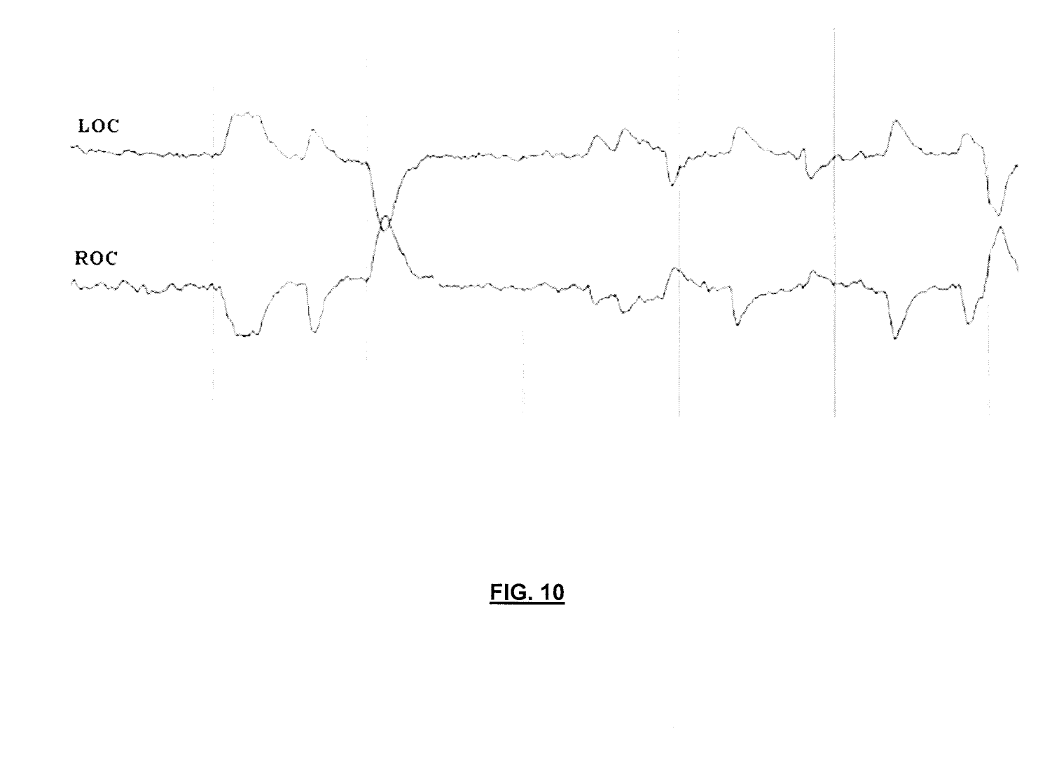

The filter is creating a zero-mean time-series. The bilateral segmentation is performing simultaneous segmentation on left and right EOG signals and produces candidate wavelets, as shown in FIG. 10. The spatial filter analyzes the field of the signal and if not of ocular origin discards the candidate wavelet.

The input time series for segmentation are all zero-mean.

We build the time series:

A[n]=loc[n]−roc[n]

where we used the channel label to denote time series obtained from the channels with the same name (e.g. loc[n] represents the n-th sample of the left oculogram).

We define some constants:

MIN_REM_A=30 uV

MIN_REM_T=140 ms

We define a candidate wavelet wave[i] as the convex set of indices:

$$wave [ i ] = \{ \begin{matrix}k \vert \vert diff [ k ] \vert > \vert loc [ k ] \vert \vert diff [ k ] \vert > \vert roc [ k ] \vert \vert diff [ k ] \vert > MIN\_REM \_A \\ ( 1 - t ) {k}_{1} + {tk}_{2} ∈ wave [ i ] ∀ k 1 ∈ wave [ i ] , k 2 ∈ wave [ i ] , t ∈ [ 0 , 1 ]\end{matrix} \}$$

The vertex of the wavelet is the index extracted by the vertex operator:

vertex(x, wave[i])=(x[wave[i]]>0)*argmax (x[wave[i]])+(x[wave[i]]<0)*argmin (x[wave[i]])

x={eogL, eogR}

In the following text, to simplify notation it will be understood that when estimating from signals on the left vertex will be of the form vertex(eogL, .) and we will simply write vertex(.). The same applies for start and end wavelet operators.

In the equation above the vertex operator extracts the vertex of the set x for the set of indices wave[i].

For each candidate wavelet we determine the noise on each side:

We build the ndx set:

$$Zx = \{ k \vert k ∈ wave [ i ] \frac{d x}{d t} [ k ] = 0 \}$$

$$where$$

$$x = \{ loc , roc \}$$

Define:

NoiseL[i]=eogL[vertex(wave[i]]−eogL[start(wave[i])]>0*max(|min{eogL[k]−eogL[k−1]|k ∈ [start(wave[i]), vertex(wave[i])}|, |max{eogL[k]−eogL[k−1]|k ∈ [vertex(wave[i]), end(wave[i])}|+eogL[vertex(wave[i]]−eogL[start(wave[i])]<0*max(max{eogL[k]−eogL[k−1[|k ∈ [start(wave[i]), vertex(wave[i])}, |min{eogL[k]−eogL[k−1]|k ∈ [vertex(wave[i]), end(wave[i])}|))

NoiseR[i]=eogR[vertex(wave[i])]−eogR[start(wave[i])]>0 *max(|min{eogR[k]−eogR[k−1]|k ∈ [start(wave[i]), vertex(wave[i])}|, |max{eogR[k]−eogR[k−1]|k ∈ [vertex(wave[i]), end(wave[i])}|+eogR[vertex(wave[i]]−eogR[start(wave[i])]<0*max(max{eogR[k]−eogR[k−1]|k ∈ [start(wave[i]), vertex(wave[i])}, |min{eogR[k]−eogR[k−1]|k ∈ [vertex(wave[i]), end(wave[i])}|))

Compute:

$$SN [ i ] = min ( \frac{eogR [ vertex ( wave [ i ] ) ]}{NoiseR [ i ]} , \frac{eogL [ vertex ( wave [ i ] ) ]}{NoiseL [ i ]} )$$

Twave[i]=end(wave[i])−start(wave[i])

Twave[i] is the duration of the i-th candidate wavelet;

A further selection of wavelets is applied as follows:

For REMs we decimate the wave set {wave[i]}:

wave[k]={wave[i]|SN[i]>MIN_{SN} [symbol] Twave[i]>MIN_{T}_{_{REM}} [symbol] vertex(eogL, wave[i])−vertex[eogR, wave[i])<MAX_{VV}}

We build the field:

source[i]=argmax(eegL(wave[i]), eegR(wave[i]), eogL(wave[i]), eogR(wave[i]))>2

We further decimate the set wave[k]:

wave[k]=wave[k]*source[k]

When source[k]=0 wave[k] is deleted.

At this point we have a set of wavelets with the right relative polarity and field. These wavelets represent the sum set of REMs during wake and REM stages of sleep.

Each epoch has a set {REMⱼ} of times where a REM occurred. These times correspond to:

REMᵢ=vertex(wave[i])

The same procedure is used to detect Slow Eye Movements (SEM) with two minor changes:

We replace MIN_REM_T with MIN_SEM_T and insert anywhere in the algorithm the conditions of wavelet symmetry:

vertex(loc, wave[i])−start(loc, wave[i])<C*vertex(roc, wave[i])−start(roc, wave[i])

vertex(roc, wave[i])−start(loc, wave[i])<C*vertex(loc, wave[i])−start(roc, wave[i])

MIN_SEM_T=600 ms

C=1.5

The whole study has a set of sets of REMS; one REM set for each epoch “j” {REMⱼ}, REMⱼ is a set of REMs in epoch “j”.

The REM density can then be estimated in multiple ways depending on the purpose. In one case, a rolling window of variable duration can be used, depending on the length of the REM episode.

$$RD [ k ] = \frac{{\sum }_{i = - \frac{M}{2}}^{\frac{M}{2}} StageREM ( k - i ) * Card ( {REM}_{k} )}{{\sum }_{i = - \frac{M}{2}}^{\frac{M}{2}} StageREM ( k - i )}$$

Setting M=1 we get REM count per epoch. Setting:

$$M ( k ) = arg max {\sum }_{i = - \frac{M}{2}}^{\frac{M}{2}} StageREM ( k - i ) ( 1 - t ) {k}_{1} + {tk}_{2} ∈ [ k - \frac{M}{2} , k + \frac{M}{2} ] ∀ k 1 ∈ [ k - \frac{M}{2} , k + \frac{M}{2} ] k 2 ∈ [ k - \frac{M}{2} , k + \frac{M}{2} ] , \\ t ∈ [ 0 , 1 ]$$

This translates to setting M to the largest possible value such that the set of REM epochs is convex. In this case we get the average REM count per REM episode, where the duration of the REM episode can be anything between one and hundreds of epochs. Stage REM(k) is 1 in case epoch k corresponds to a REM stage, 0 otherwise.

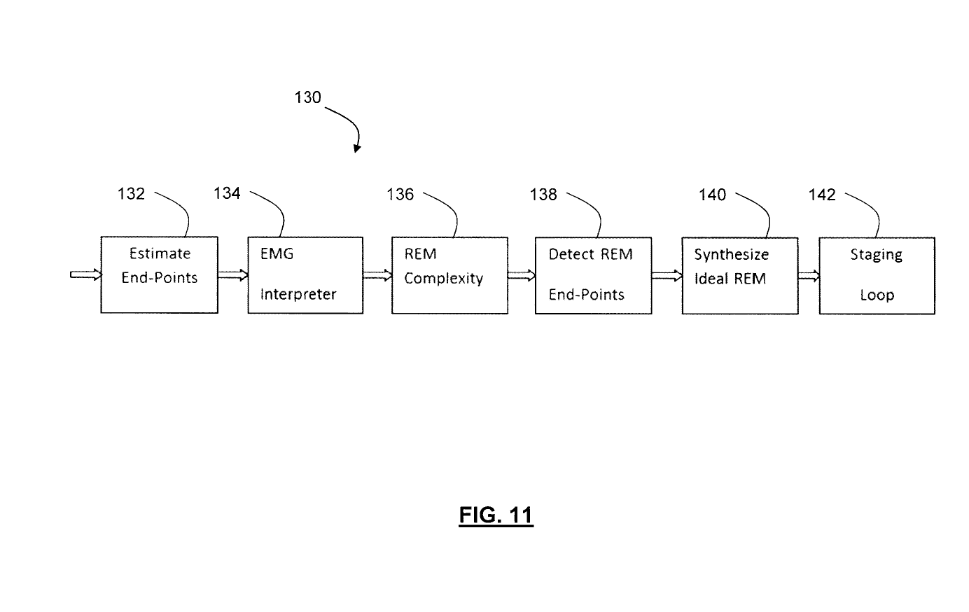

The system 100 also includes a stager 130. One embodiment of a stager 130 is shown in greater detail in FIG. 11.

The input to the stager 130 is a time series of state vectors containing epoch descriptors (see FIGS. 5 and 11).

$$state [ i ] = [ \begin{matrix}cmplx [ i ] \\ emgL [ i ] \\ emgR [ i ] \\ emgC [ i ] \\ REMD [ i ] \\ SEMD [ i ] \\ domfL [ i ] \\ domfR [ i ]\end{matrix} ] ,$$

state[i] represents the state vector of epoch “i”. The complexity, cmplx[i] represents the length of the shortest code that can encode the epoch and permit reproduction without any loss.

In FIG. 11, one can follow the operations needed to perform staging. This module will be described in broad outline and then with details for each module.

Due to patient variability and variable noise conditions, analysis is automatically calibrated for each patient. Thus, these techniques may not generally present a real-time approach, although adaptations of the method for such real-time applications are conceivable.

The state of consciousness of the patient is a continuum while the sleep stages used in clinical practice are discrete. Breaking the continuum into discrete states requires setting state boundaries. We will refer to the process of determining these boundaries as End-Point detection. Sometimes determining the End Points is not simple and can represent a source of error.

The EMG Interpreter 134 determines the representative EMG level for Wake, Sleep and REM that are useful for classifying ambiguous states or short transients.

The REM complexity module 136 establishes the plateau of REM state in the light of complexity and establishes the REM EMG levels using information from the EMG analyzer.

Having established the REM EMG and the REM complexity, one then determines the REM end points (i.e., using the Detect REM End Points module 138).

Having determined the REM end-points, in order to detect REM episodes that have no detected REMs, one can then synthesize an ideal REM 140 based on the REM episodes detected so far. After the REM episodes have been identified we enter the staging loop 142 and perform the staging of the whole study, epoch-by-epoch using the end-points detected earlier.

The estimate end-points module 132 is generally quite important to the stager 130 and errors at this point can be catastrophic for the performance of the stager 130. The input state vectors are accurate and very reliable. Determining the end points can be a critical step of the staging. While the complexity is an accurate continuous reflection of the continuous patient state, determining the end points accurately is important in order to establish agreement with the current practice of sleep staging, which uses discrete states.

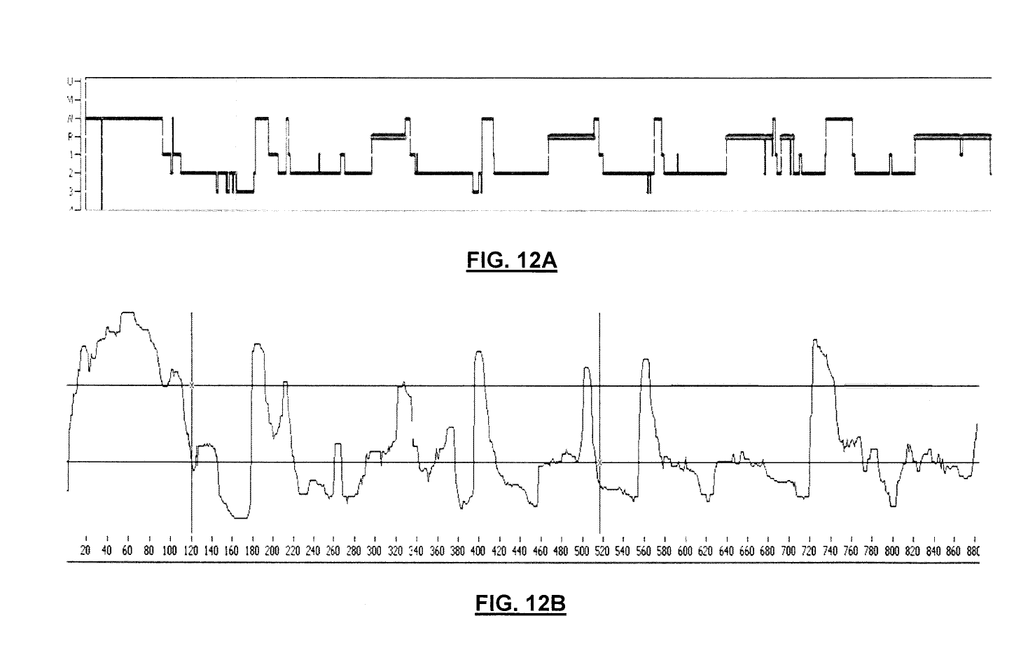

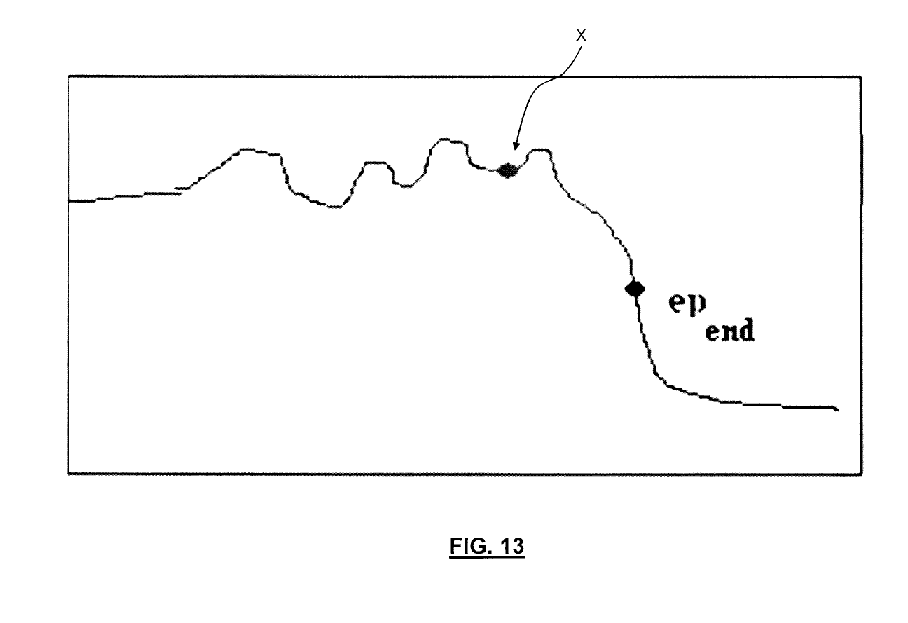

In FIGS. 12A and 12B, one can see the correlation of the sleep stages as determined by a human reviewer (FIG. 12A) and the complexity of the EEG estimated using the teachings herein (FIG. 12B). Clearly the EEG generated according to the teachings herein follows the stages marked by the human reviewer. This module establishes the boundaries between stages W-S1, S1-S2 and S2-S3.

While one may not know the exact end-points for each patient, in general the end points are fairly stable with some exceptions. In order to include the exceptions, in some embodiments the technique can be modified to get the generality useful across age groups and treatment regimens and conditions.

The end-point calculation starts by finding a point in time that falls definitely during sleep, we call that epoch ep_{end}.

ep_{end}={min(i)|cplx[i]<DB [symbol] cplx[i]<cplx[deepndx]+0.02; i ∈ [1, N]; deepndx=argmin(cplx[i]=□_{0.1}{cplx[i]})

DB=0.76.

Next we detect the W-S1 boundary. Empirical observation leads us to the conclusion that looking backward from ep_{end} we set the highest local minimum before falling asleep as the minimum characteristic complexity for W.

$$WS 1 = max ( cmplx [ i ] ) \vert i < {ep}_{end} , \\ \frac{d cplx}{d t} [ i ] = 0 , \\ \frac{{d}^{2} cplx}{d {t}^{2}} [ i ] > 0$$

Next we detect the S1-S2 boundary.

Here we have two cases or classes of subjects or patients: the case of alpha generators and not alpha generators.

The alpha generators are individual patents that have enough alpha activity on the EEG to help distinguish the wake state based on alpha. For the alpha generators there is a landmark that marks the transition from S1-S2 based on dominant frequency. The complexity drops abruptly and the dominant rhythm falls from above 7 Hz to below 7 Hz.

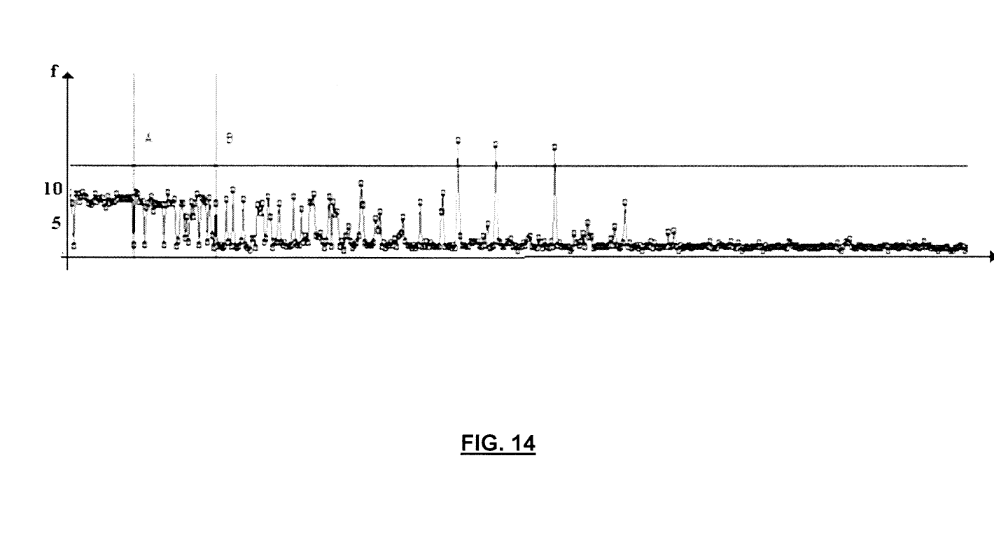

In FIG. 14 we note at B the transition from S1-S2 (B) and W-S1 (A). The characteristic of the transition is the switching of dominant frequency from very low to above 7 Hz. We call this region a region of bistability (switching between two states). Once the state settles, bistability disappears and one of the states “Wake” or “S2” becomes the clear pattern. The region with dominant frequency below 5 Hz is S2 and above 5 Hz is S1.

WS1=cplx[max(i)]|card({domfLⱼⁱ5 <5})=0 [symbol] card({domfRⱼⁱ<5})=0, j ∈ [1,10], i<ep_{end}) S1S2=cplx[max(i)]|card({domfⱼⁱ>5})=card({domfⱼⁱ<5}), j ∈ [1,10], i<ep_{end})

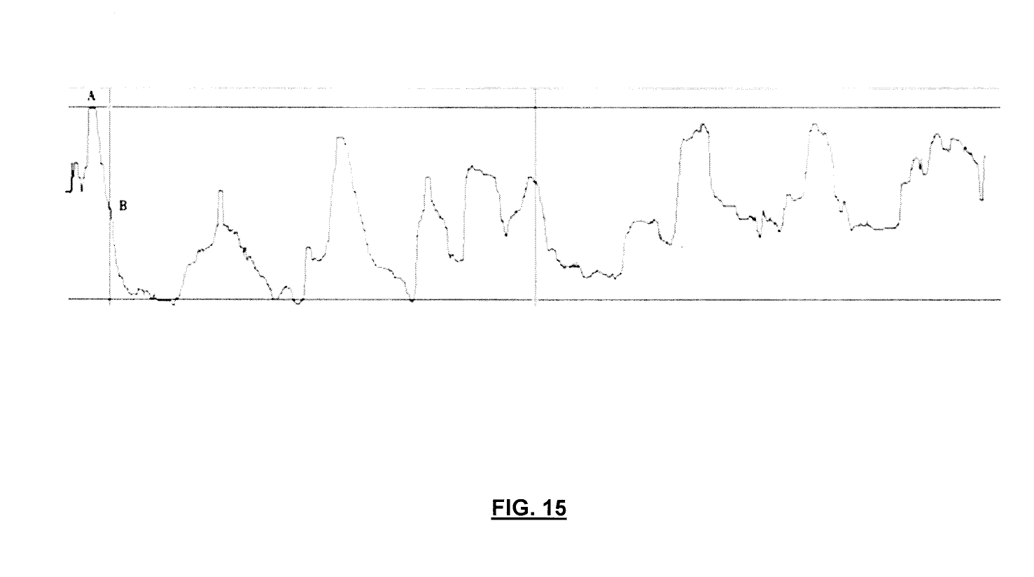

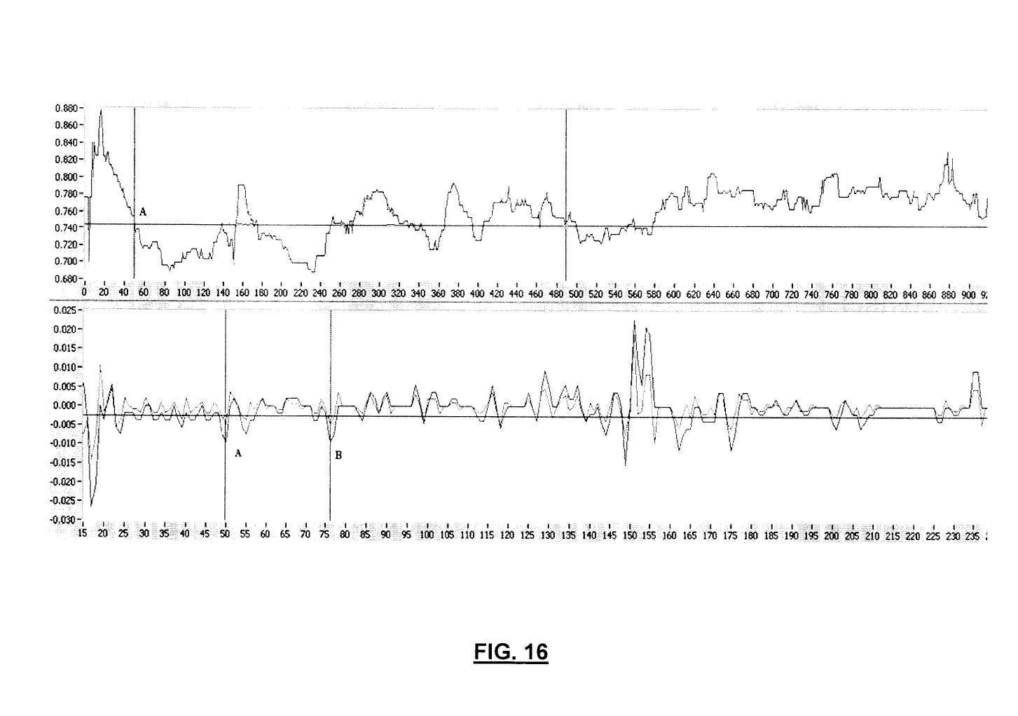

However, for patients that are not alpha generators, another mechanism is used to distinguish the wake state. First we determine the point where beta had a local maximum before it dropped to ½ (see point A in FIG. 15) from the last maximum value before sleep onset.

$${beta}_{H} = arg max ( \frac{d beta [ k ]}{d t} = 0 \frac{{d}^{2} beta [ k ]}{d {t}^{2}} < 0 , k < {ep}_{end} )$$

$$S 1 S 2 = cplx ( arg min ( \frac{d cplx [ k ]}{d t} > - 0.003 ) ) ;$$

$$k > arg min ( \frac{d cplx [ i ]}{d t} < - 0.008 ) , \\ {beta}_{H} < i < {ep}_{end}$$

The S1-S2 transition corresponds to the value of the complexity at a minimum negative change of complexity of 0.008/epoch between the point beta_{0.5} and the upper boundary of S3.

The onset of sleep is considered to be the earliest drop in information content (complexity) under the level of the boundary S1/S2.

The S2-S3 boundary is empirically determined to be the 98 percentile of the complexity corresponding to an epochal probability of delta increased by 20% relative to the median delta during the whole sleep record excluding the periods when patient is awake.

We build the sets of epochal delta estimates (D) and epochal complexities corresponding to an increased delta by 20% relative to the sleep median (C).

D={delta[i]; cplx[i]<WS1)

C={cplx[i]|delta[i]>0.2+□_{0.5}D}
The □ₚ represents the rank p set operator.

p=0.98*card(C),

S2S3=□pC

At this point we have estimated all necessary boundaries (WS1, S1S2, S2S3)).

The EMG interpreter module 134 analyzes the EMG activity on all channels (A1, A2, CHIN1-CHIN2) and outputs the representative levels of skeletal muscle tone for wake (W), non REM (NREM) and REM sleep (REM) according to the following algorithm:

wemgL=□_{0.5}{emgL[i]|cplx[i]>WS1}

wemgR=□_{0.5}{emgR[i]|i<argmin(cplx[i]>WS1)}

wemgC=□_{0.5}{emgC[i]|i<argmin(cplx[i]>WS1)}

We consider the sleep onset the epoch where cplx[i]<S1S2 the first time.

At the same time we calculate the alpha dominant during Wake:

alphaWL=mode{alphaL[i]|i<onset}

alphaWR=mode{alphaR[i]|i<onset}

alphaWX=mode{alphaX[i]|i<onset}

slemgL=mode{emgL[i]|cplx[i]<S1S2}

slemgR=mode{emgR[i]|cplx[i]<S1S2)}

slemgC=mode{emgC[i]|cplx[i]<S1S2)}

emgremL=□_{0.5}{emgL[i]|cplx[i]<S1S2, emgL[i]<0.8*wemgL}

emgremR=□_{0.5}{emgR[i]|cplx[i]<S1S2, emgR[i]<0.8*wemgR}

emgremC=□_{0.5}{emgC[i]|cplx[i]<S1S2, emgC[i]<0.8*wemgC}

studyfmodeL=mode{domfL[i]|cplx[i]<S1S2}

studyfmodeR=mode{domfR[i]|cplx[i]<S1S2}

studyfmodeX=mode{domfX[i]|cplx[i]<S1S2}

The REM complexity module 136 estimates the complexity (information) of REM sleep. First a preliminary REM boundary detection is performed based on maximum REM EMG levels established by the EMG Interpreter and complexity associated to detection of rapid REMs. Next the candidates are recursively tested against the minimum EMG REM episode and episodes with largely different EMG will be deleted. The highest density REM will be used as a robust representative of REM EMG and REM complexity.

REM boundaries are established by finding epochs with nonzero REM density and ending when either skeletal EMG tone is increased or due to presence of spindles or complexity swings larger than 2% relative to the complexity from the start of the episode.

The REM density calculation is essentially an average REM count in the window between the first and last REM epoch. The important aspect is that the individual REMs are validated against potential arousals coincident or succeeding the REMs. This is necessary as the set of originally detected REMs correspond either to Wake, REM or Arousals.

A Boolean function checks if there is a power jump in the band higher than alpha during W minus 1 Hz (powalpha[t]):

isArousal[i]=(t−REMᵢ<3fs) [symbol] powalpha[t]>max{powalpha[k]|k ∈ [t−10, t−6]}

start[i]=i|RD>0 [symbol] emgC[i]<k*emgremC [symbol] cplx[i]<WS1 [symbol] (emgC<0.8wemgC [symbol] cplx[i]<S1S2+0.02)

end[i]=min(j)|RD=0 [symbol] (emgC[j]>k*emgremC [symbol] |cplx[j]−cplx[start[i]|>0.02)

The i-th REM episode boundaries are:

$$REM [ i ] = [ \begin{matrix}start [ i ] \\ end [ i ]\end{matrix} ]$$

Next we delete the REM episodes with high skeletal tone:

REM[i]={REM[i]|□_{0.5}{emgL[i]|start[i]<i<end[i]}<2*minemgL [symbol] □_{0.5}{emgR[i]|start[i]<i<end[i]}<2*minemgR [symbol] □_{0.5}{emgC[i]|start[i]<i<end[i]}<2*minemgC}

cplx_{REM}=□_{0.5}{cplx[k]|k ∈ ∪ᵢ[start[i], end[i]]

emgremL=□_{0.8}{emgL[k]|k ∈ ∪ᵢ[start[i], end[i]]}}

emgremR=□_{0.8}{emgR[k]|k ∈ ∪ᵢ[start[i], end[i]]}

emgremC=□_{0.8}{emgC[k]|k ∈ ∪ᵢ[start[i], end[i]]}

The REM boundaries module 138 is a second iteration of the REM boundary detection described above but using the refined parameters estimated therein.

The boundaries are adjusted using the conditions or narrow information (complexity) swing during REM sleep, keeping in mind the convexity of the set as follows:

start[i]=min(j)|cplx[j]−cplx[start[i]<0.02 [symbol] emgC[j]<k*emgremC [symbol] (1−t)k₁+tk₂ ∈ REM[i] ∀ k1 ∈ [j, start[i]], k2 ∈ [j, start[i]], t ∈ [0,1]

end[i]=max(j)||cplx[j]−cplx[start[i]<0.02 [symbol] emgC[j]<k*emgremC [symbol] (1−t)k₁+tk₂ ∈ REM[i] ∀ k1 ∈ [end[i], j], k2 ∈ [end[i], j], t ∈ [0,1]|

K=1.6;

At the “Synthesize Ideal REM Module” 140, we have a number of detected REM episodes and we are trying to detect ones that might have been not detected due to null REM density or failure to detect REMs due to various reasons (e.g. loose EOG electrode unilaterally).

$${cplxrem}_{id} = {•}_{0.5} \{ cplx [ k ] \vert k ∈ {⋃}_{i} [ start [ i ] , end [ i ] ] \\ {emgremL}_{id} = {•}_{0.2} \{ emgL [ k ] \vert k ∈ {⋃}_{i} [ start [ i ] , end [ i ] ] \} \\ {emgremR}_{id} = {•}_{0.2} \{ emgR [ k ] \vert k ∈ {⋃}_{i} [ start [ i ] , end [ i ] ] \} \\ {emgremC}_{id} = {•}_{0.2} \{ emgC [ k ] \vert k ∈ {⋃}_{i} [ start [ i ] , end [ i ] ] \}$$

This is similar to Estimate REM Complexity, but with stricter rules on EMG values and no need for the recursion as we are at this point generally certain that the REM set is accurate.

The staging loop module 142 then goes epoch-by-epoch and outputs the corresponding stage.

$$stage [ i ] = [ \begin{matrix}rem [ i ] \\ w [ i ] \\ s 1 [ i ] \\ s 2 [ i ] \\ s 3 [ i ] \\ s 4 [ i ]\end{matrix} ]$$

Only one of the elements of the Boolean vector is non zero. The element of the stage[i] vector is a Boolean function.

The i-th REM episode boundaries are:

$$REM [ i ] = [ \begin{matrix}start [ i ] \\ end [ i ]\end{matrix} ]$$

The Boolean function:

$$rem [ i ] = ( i - start [ k ] ) ( end [ k ] - i ) > 0 + ( emgC [ i ] < k * {emgremC}_{id} ) * ( \vert cplx [ i ] - {cplxrem}_{id} \vert < 0.01 ) * ( emgC [ i ] < 0.8 wemgC ) * ( {cplxrem}_{id} > 0 ) * ( \vert \frac{d cplx}{d t} [ i ] \vert < 0.005 )$$

Epoch I will be staged as REM if the epoch number falls within the boundaries of the i-th REM episode with boundaries REM[i] or the complexity is within a band not more than 1% from the ideal REM complexity and the skeletal muscle tone is characteristic to REM. At the same time we exclude transitory states, namely the complexity must be stationary and there must be at least one epoch with non zero REM density.

The rest of the Boolean functions are:

s3[i]=(cplx[i]<S2S3)

s4[i]=(cplx[i]<S3S4)

w[i]=(cplx[i]>WS1)*(emgL[i]>k*wemgL+emgR[i]>k*wemgR+emgL[i]>k*wemgL≧2)

s2[i]=(cplx[i]<S1S2)

s1[i]=(cplx[i]>S1S2)

### Discussion

Presented below are the results of tests our 107 adult patients and 25 youth patients (under 18 years of age). The results are grouped this way due to the different patient groups available, however an overall value can be computed easily using a weighted average, considering the relative number of epochs of the group as weights.

It is clear from the tables (Table 1-10) that the results on agreement are tightly grouped around 80%. In particular, the overall sensitivity epoch-by-epoch agreement is better than 80%. The overall sensitivity per stage agreement is approximately 80%.

TABLE 1
Results on the set ADC (14 patients). Overall agreement 81%.

| Detector | Sensitivity | Total TP | Total FP | Total FN | Total Epochs |
|---|---|---|---|---|---|
| REM | 0.866887 | 2097 | 262 | 322 | 12672 |
| SD | 0.783615 | 1253 | 445 | 346 | 12672 |
| SL | 0.836145 | 5205 | 1093 | 1020 | 12672 |
| Wake | 0.775216 | 1883 | 434 | 546 | 12672 |

TABLE 2
Results on the set ADD (12 patients). Overall agreement 82%.

| | | | | | Total |
|---|---|---|---|---|---|
| Detector | Sensitivity | Total TP | Total FP | Total FN | Epochs |
| REM | 0.785924 | 1072 | 218 | 292 | 11237 |
| SD | 0.686654 | 355 | 220 | 162 | 11237 |
| SL | 0.845867 | 6344 | 690 | 1156 | 11237 |
| Wake | 0.793103 | 1472 | 866 | 384 | 11237 |

TABLE 3
Results on the set LFT (10 patients). Overall agreement 80%.

| | | | | | Total |
|---|---|---|---|---|---|
| Detector | Sensitivity | Total TP | Total FP | Total FN | Epochs |
| REM | 0.800611 | 1048 | 191 | 261 | 9708 |
| SD | 0.778455 | 766 | 285 | 218 | 9708 |
| SL | 0.810006 | 4404 | 787 | 1033 | 9708 |
| Wake | 0.814965 | 1612 | 615 | 366 | 9708 |

TABLE 4
Results on the set SFRV (41 patients). Overall agreement 80%.

| Detector | Sensitivity | Total TP | Total FP | Total FN | Total Epochs |
|---|---|---|---|---|---|
| REM | 0.823568 | 5046 | 1340 | 1081 | 36442 |
| SD | 0.808756 | 2660 | 1509 | 629 | 36442 |
| SL | 0.790137 | 16743 | 2555 | 4447 | 36442 |
| Wake | 0.788211 | 4600 | 1989 | 1236 | 36442 |

TABLE 5
Results on the set SFR (13 patients). Overall agreement 81%.

| | | | | | Total |
|---|---|---|---|---|---|
| Detector | Sensitivity | Total TP | Total FP | Total FN | Epochs |
| REM | 0.840122 | 1650 | 402 | 314 | 11319 |
| SD | 0.824924 | 1079 | 374 | 229 | 11319 |
| SL | 0.811699 | 5384 | 751 | 1249 | 11319 |
| Wake | 0.774399 | 1095 | 584 | 319 | 11319 |

TABLE 6
Results on the set SLV (10 patients). Overall agreement 82%.

| Detector | Sensitivity | Total TP | Total FP | Total FN | Total Epochs |
|---|---|---|---|---|---|
| REM | 0.85662 | 1404 | 211 | 235 | 9067 |
| SD | 0.868936 | 1021 | 525 | 154 | 9067 |
| SL | 0.810861 | 3897 | 638 | 909 | 9067 |
| Wake | 0.763649 | 1105 | 266 | 342 | 9067 |

TABLE 7
Results on the set SL (7 patients). Overall agreement 83%.

| | | | | | Total |
|---|---|---|---|---|---|
| Detector | Sensitivity | Total TP | Total FP | Total FN | Epochs |
| REM | 0.9273 | 625 | 253 | 49 | 6479 |
| SD | 0.883756 | 593 | 263 | 78 | 6479 |
| SL | 0.76459 | 2319 | 357 | 714 | 6479 |
| Wake | 0.866254 | 1820 | 249 | 281 | 6479 |

TABLE 8
Results on the set KCB (15 patients). Overall agreement 80%.

| Detector | Sensitivity | Total TP | Total FP | Total FN | Total Epochs |
|---|---|---|---|---|---|
| REM | 0.862003 | 2686 | 936 | 430 | 15596 |
| SD | 0.885948 | 3247 | 669 | 418 | 15596 |
| SL | 0.763332 | 5554 | 1320 | 1722 | 15596 |
| Wake | 0.621183 | 956 | 228 | 583 | 15596 |

TABLE 9
Results on the set KD (6 patients). Overall agreement 78%.

| | | Total | | | |
|---|---|---|---|---|---|
| Detector | Sensitivity | Tp | Total FP | Total FN | Total Epochs |
| REM | 0.765835 | 1197 | 198 | 366 | 6227 |
| SD | 0.962832 | 1088 | 480 | 42 | 6227 |
| SL | 0.761416 | 2301 | 550 | 721 | 6227 |
| Wake | 0.572266 | 293 | 120 | 219 | 6227 |

TABLE 10
Results on the set KT (4 patients). Overall agreement 88%.

| | | | | | Total |
|---|---|---|---|---|---|
| Detector | Sensitivity | Total TP | Total FP | Total FN | Epochs |
| REM | 0.931155 | 798 | 182 | 59 | 4645 |
| SD | 0.968581 | 894 | 159 | 29 | 4645 |
| SL | 0.823807 | 1744 | 168 | 373 | 4645 |
| Wake | 0.854278 | 639 | 61 | 109 | 4645 |

In addition to the epoch-by-epoch statistics, the error of final reported parameters was quantified as a result of the epoch-by epoch error. The error is described in either percent error or in absolute error, depending on what is more relevant (e.g., the error in latency is absolute error, while the error in TST is relative error). The error histograms described below are generated to inform about the error distribution in the sample.

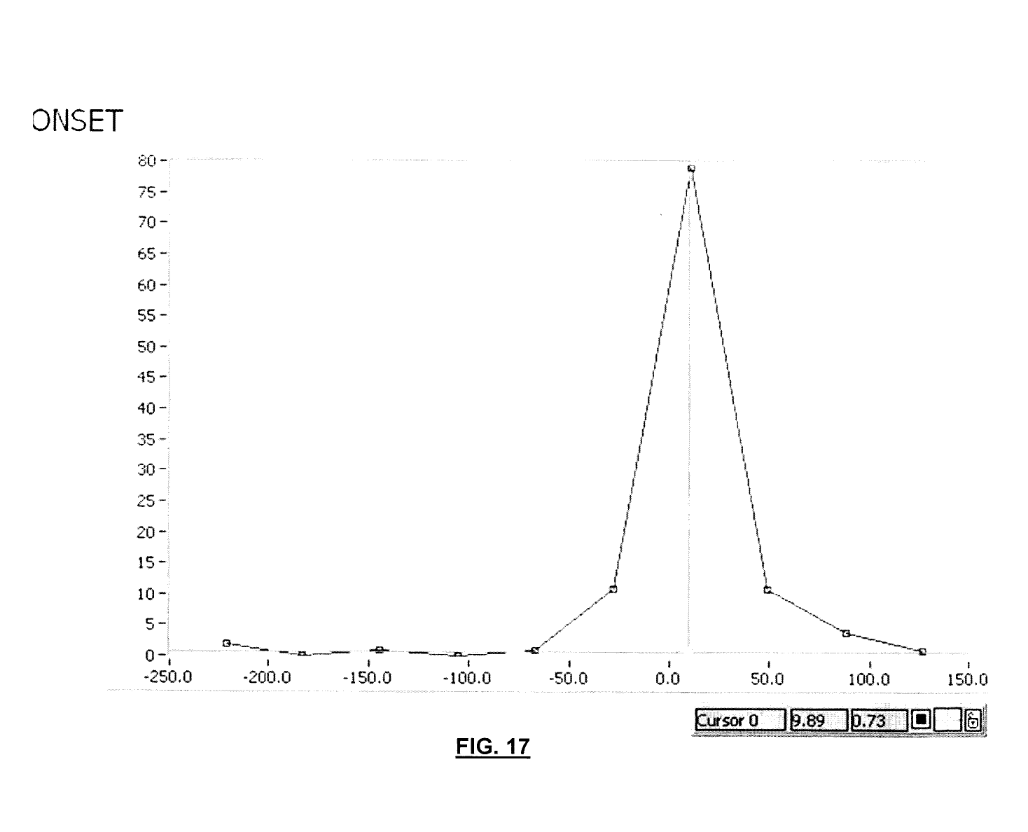

In FIG. 17, one observes that 80% of the cases have the sleep onset determined within +/−10 epochs.

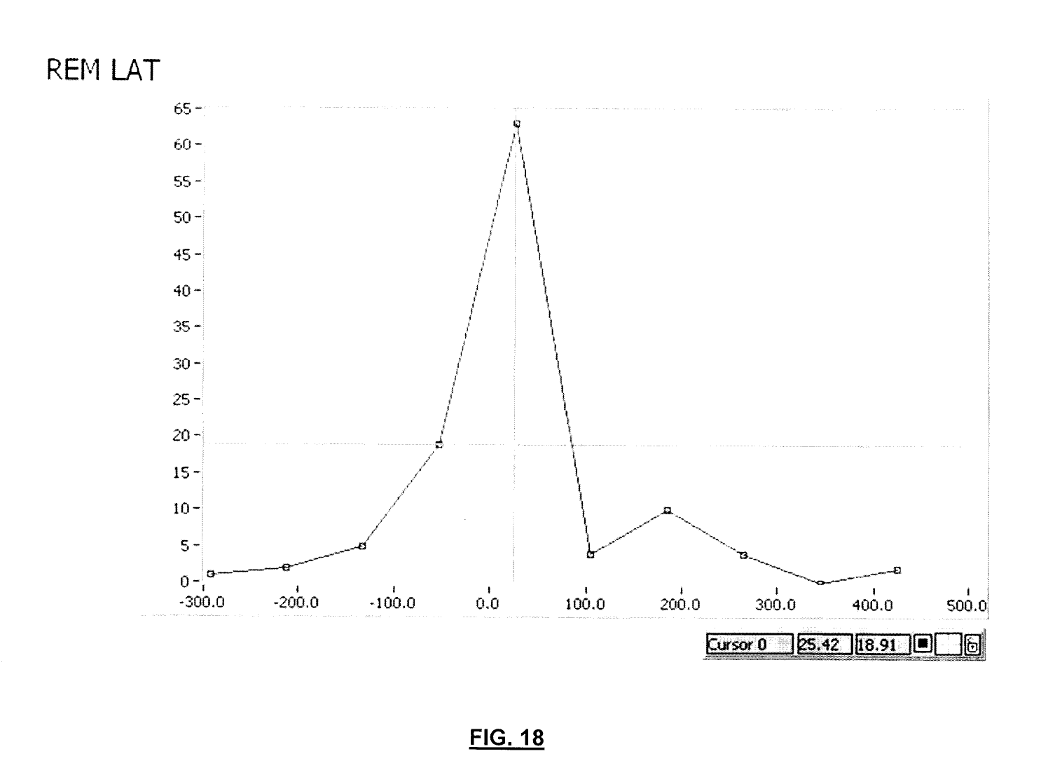

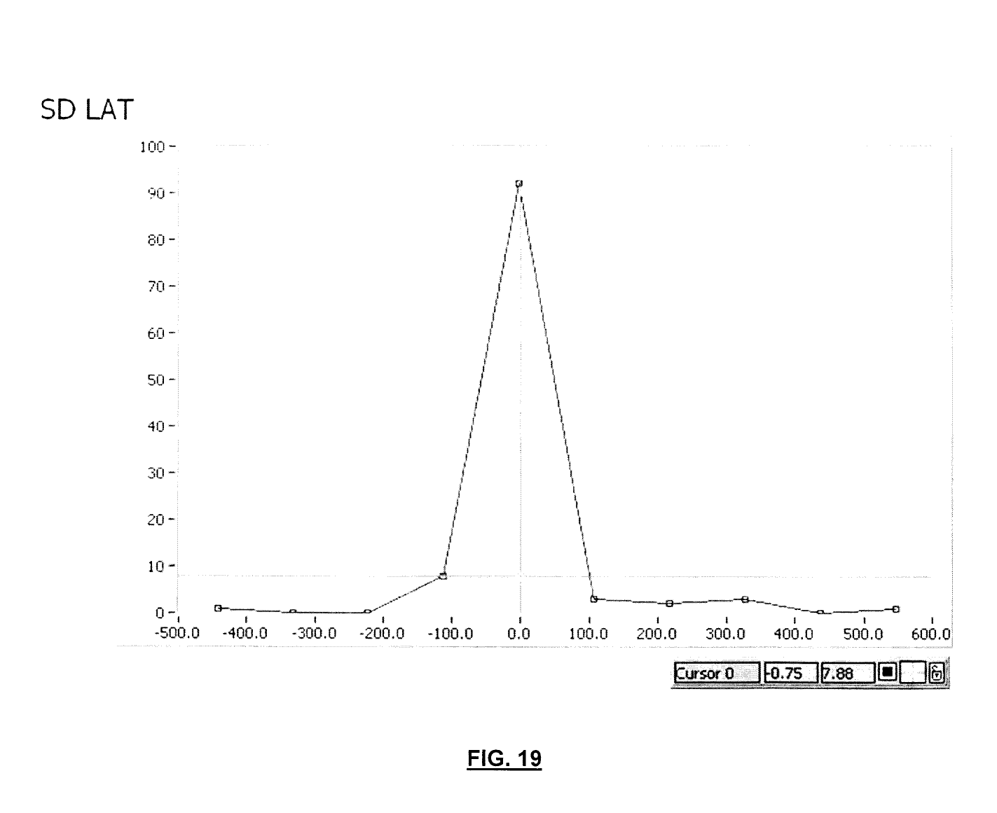

In FIG. 18, one observes that the REM latency is within +/−10 minutes 65% of the time and +−25 minutes in 85% of the cases.

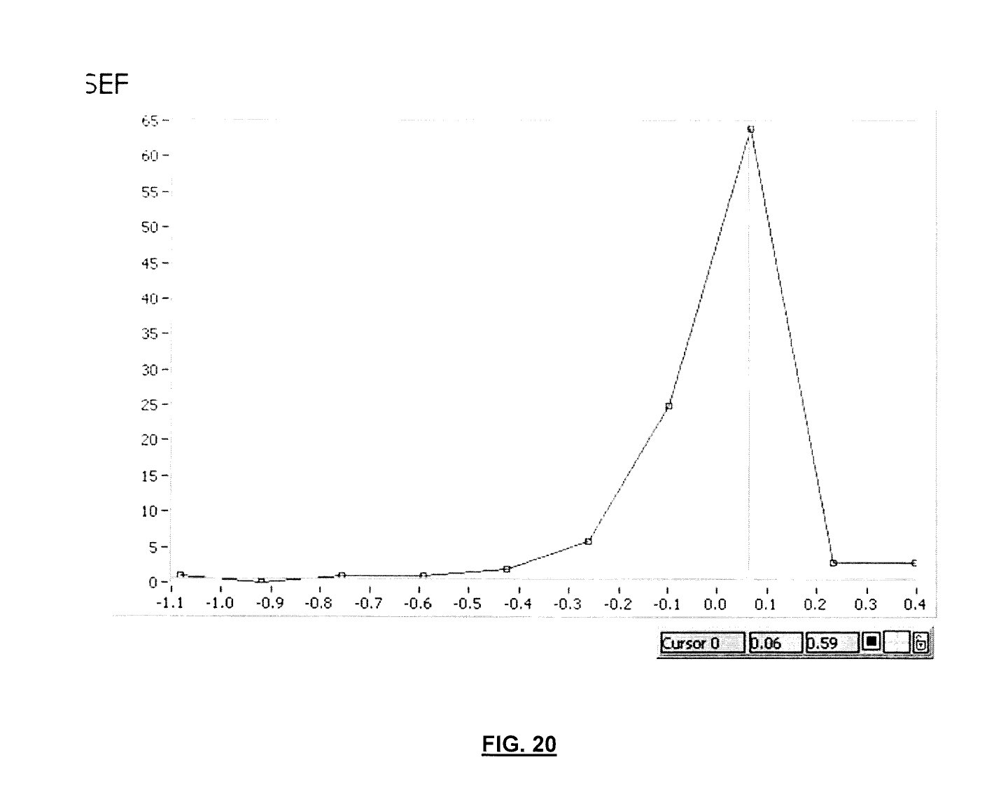

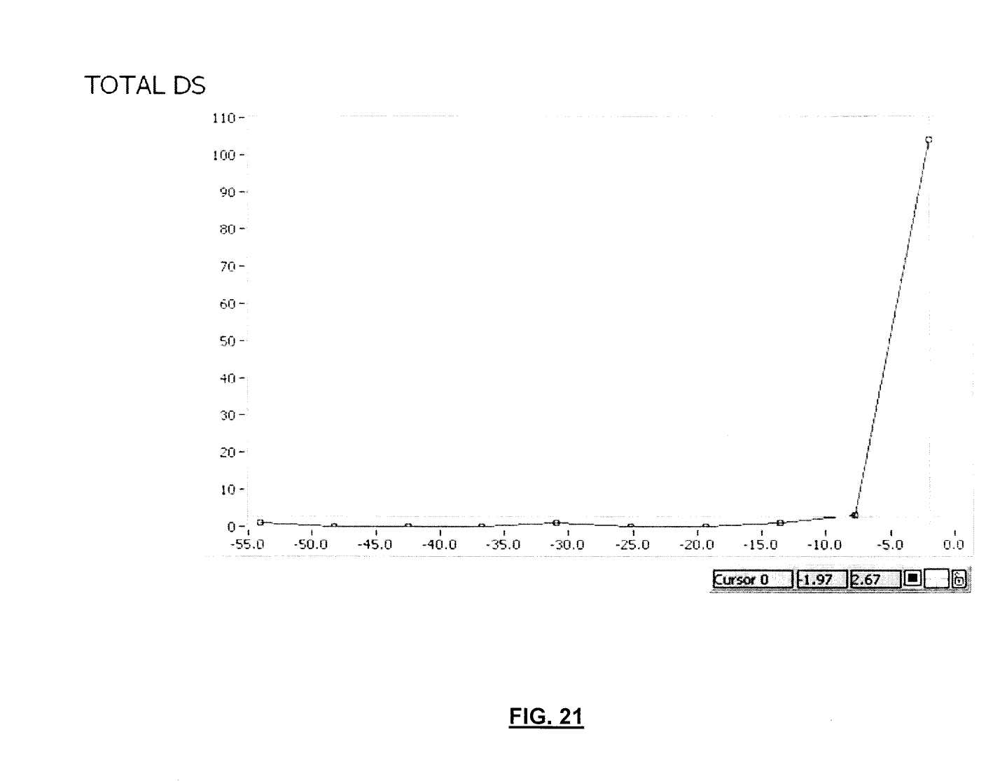

In FIG. 19 it is noted that the onset of deep sleep is exactly determined (0 latency) in over 90% of the time. The error in determination of sleep efficiency (FIG. 20) is less than 10% in over 90% of cases.

The error in the total deep sleep in the study is less than 3% in 104 cases out of 107. The LS error is due to error in S1 and S2, and is caused in general by error in REM boundaries and DS boundaries. The error in LS is less than 10% in over 75% of cases.

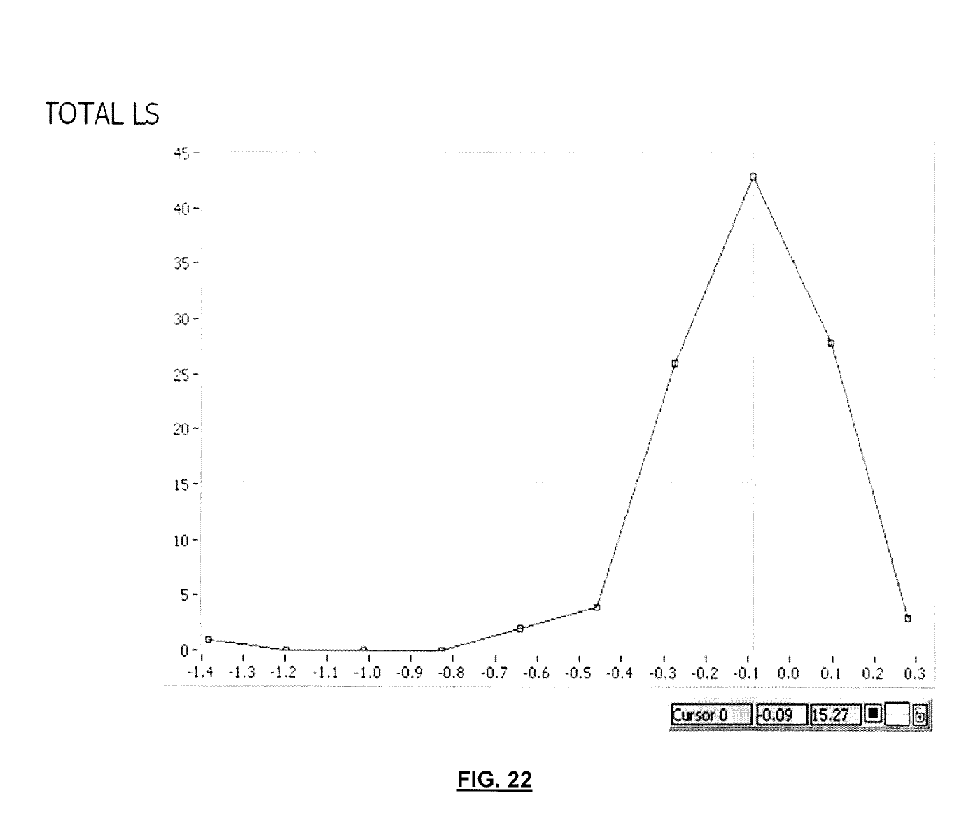

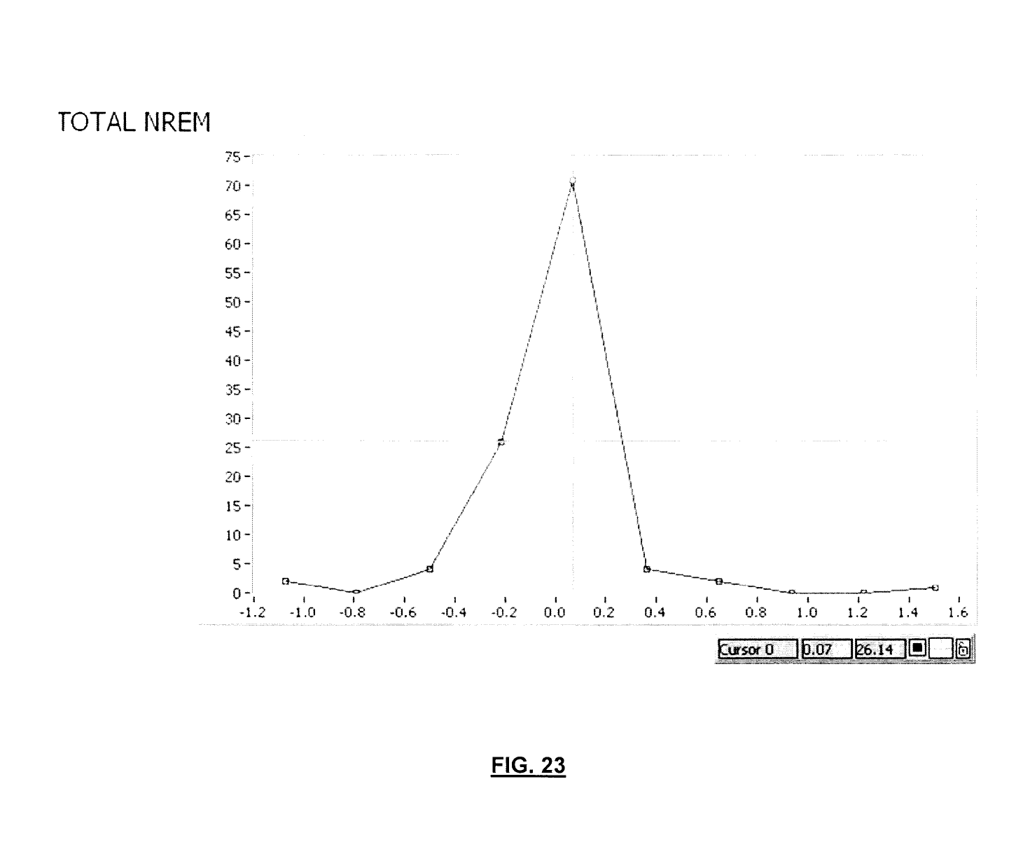

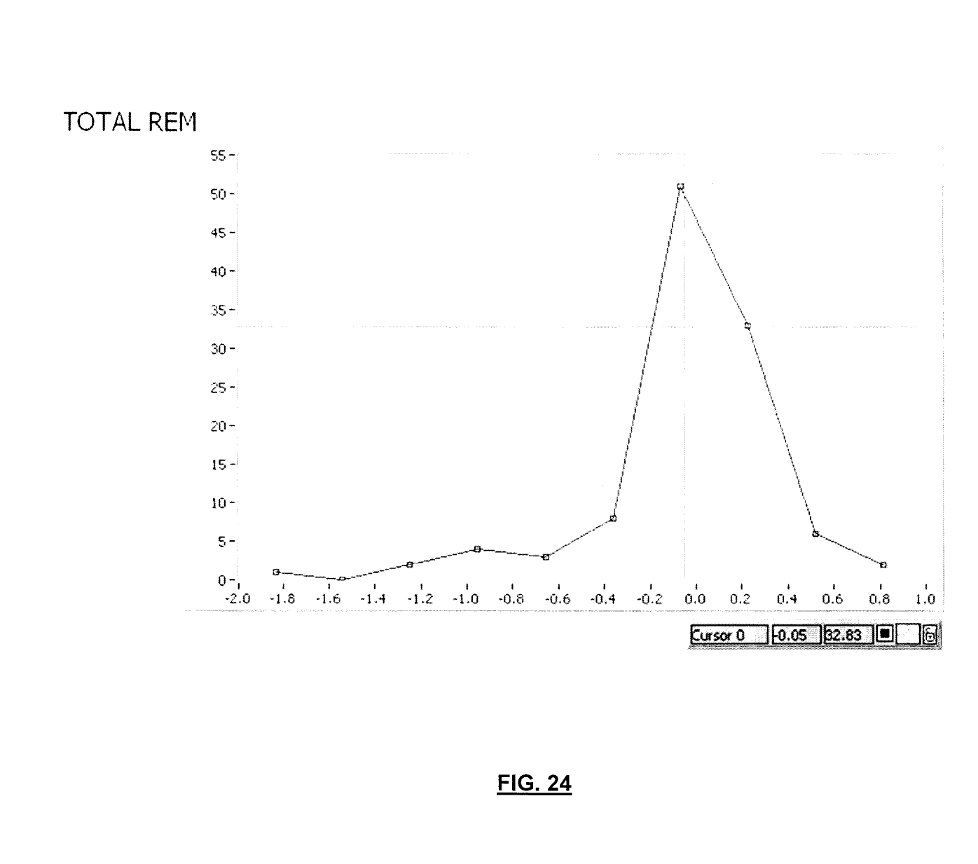

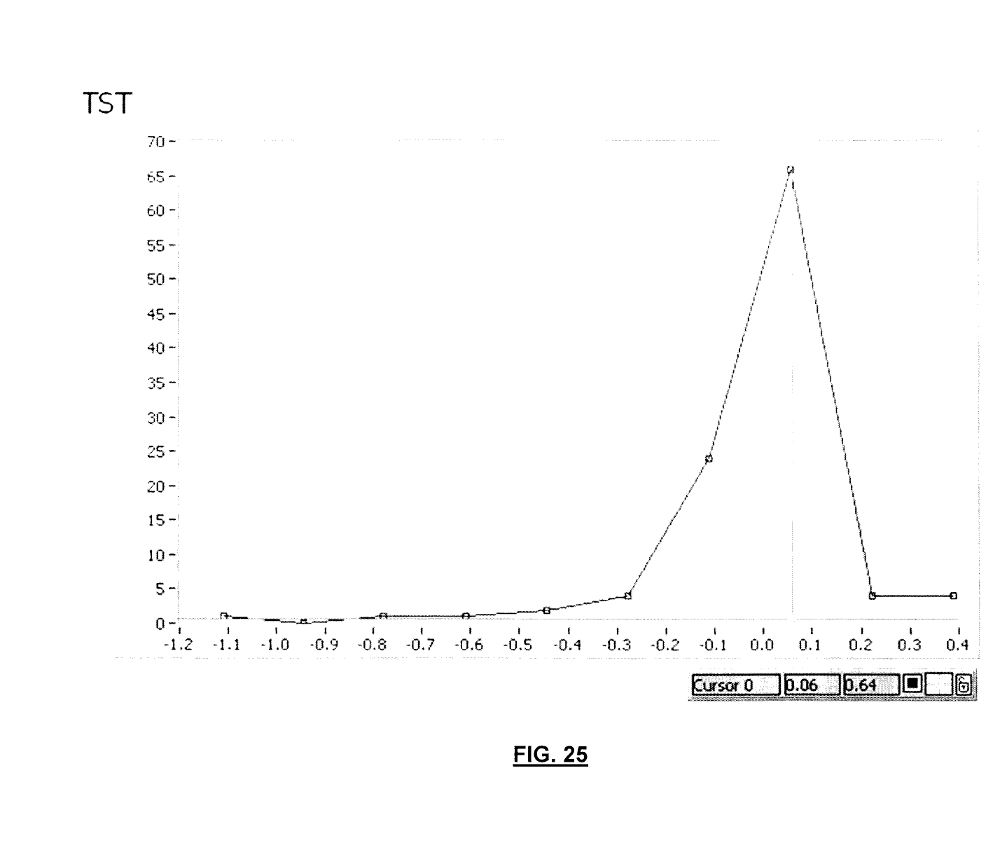

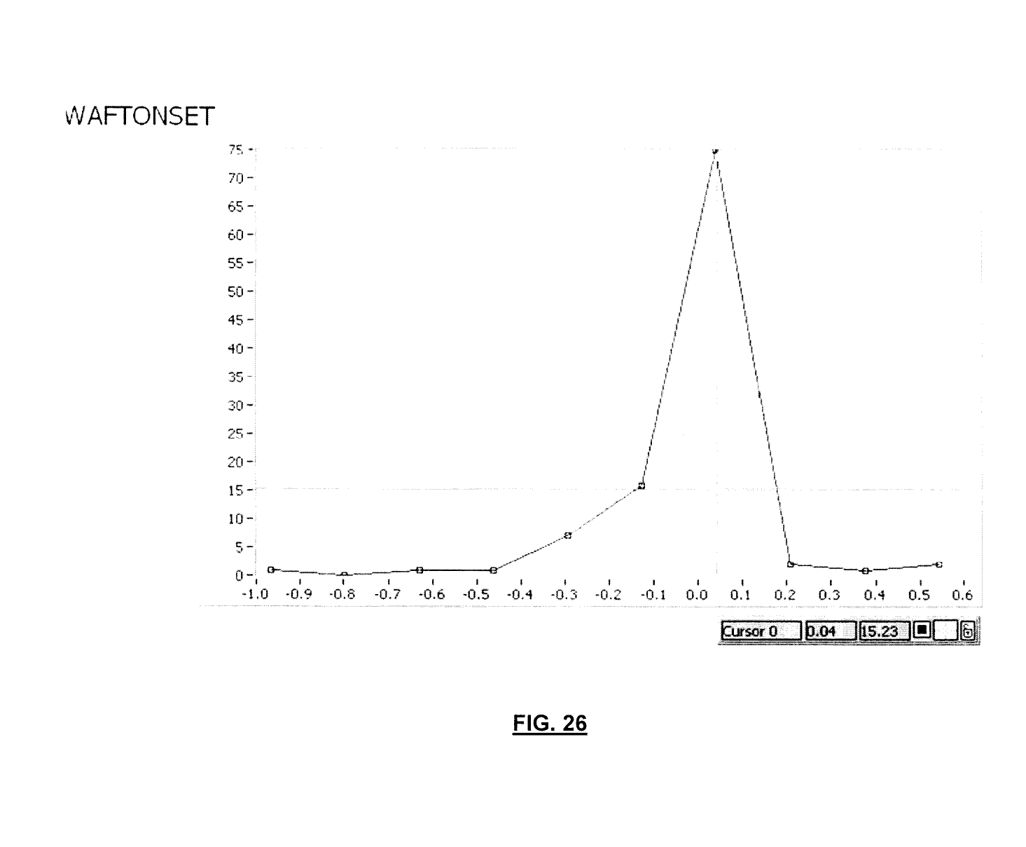

The total NREM sleep is estimated better than 80% in over 95% of cases (FIG. 23). The REM error is less than 20% in over 80% of cases (FIG. 22). The total sleep time (TST) is estimated with an error less than 10% in 90% of cases (FIG. 25). The wake after onset is estimated with an error less than 10% in 90% of cases (FIG. 26). It is believed based on these results that the systems and methods as described herein are capable of performing unattended sleep diagnostics.

It should be noted that in the marked contradistinction to the current “gold standard” of sleep diagnosis (i.e. an experienced polysomnographer applying to the best of his or her ability a set of relatively arbitrary rules), the systems and methods as described herein tend to have very clearly defined criteria and should have relatively good (and indeed potentially perfect) reproducibility from one occasion to another.

Moreover, systems and methods according to these teachings may have the advantage of objectivity, whereas the human scorer is much more susceptible to the vagaries of how particular sleep architecture features cluster.

Thus, the teachings of the present application tend to provide a truly objective algorithm while retaining the advantage of a high (but not perfect) correlation with what best human scoring can provide. It is expected that over time, the techniques described herein may become widely adopted and have the potential of becoming the de facto standard for sleep diagnosis.

Some of the teachings herein may lead to one or more advantages over conventional sleep diagnosis techniques, such as a simplified patient setup, convenience to the patient, significant cost reduction of sleep determination tests, permitting implementation in a patient's home, allowing a patient to sleep at home during testing, no need for patient's to take days off from work, no or reduced travel expenses for the patient, simplified laboratory setup and laboratory costs, reduction in cost to healthcare systems, no or reduced waiting times for lab availability, and wider coverage of the population.

At least some of these advantages may be related to the new electrode placement pattern described above with respect to FIG. 2 and from techniques that are capable of estimating a hypnogram from the new electrode data.

In particular, as shown in FIG. 2 there are no more electrodes on the scalp (as compared to conventional systems) while cerebral electrodes may simply be clipped onto the patient's ears (and which could be wireless), an operation easily performed unattended by the patient in a few seconds (as compared to the standard method that requires measurement using tape and precise positioning of electrodes on the scalp). In contrast, the conventional operation of electrode placement is time consuming due to the electrode impedance considerations and the pilosity in areas where the electrodes had to be applied.

The teachings herein may provide one or more advantages for a patient. For instance, the patient may have no need to go through a long inconvenient setup, there may be no need to sleep away from home, no waiting time due to lab appointments, no days off from work, and no travel expenses that the patient might otherwise incur.

In some cases, the teachings herein may provide at least one other benefit, namely improved safety.

In particular, physicians have oftentimes identified existing threats and led policy makers to implement regulatory approaches to reduce the associated risks. For example, respirologists have led the way in raising awareness of the threat of smoking and facilitated the process that policy makers to implement measures for the reduction of smoking.

By similarly recognizing sleep loss and sleepiness increasingly as threats and as safety risks, new policies could be implemented to help meet the corresponding challenges. Enforcing such new policies may be possible using some of the systems and methods as described herein to track compliance.

For example, driving when sleepy can be as dangerous as driving after consuming alcohol. Having a system that can automatically monitor sleepiness during driving could be extremely beneficial.

In another embodiment, the teachings herein might be useful to detect other forms of mental impairment, such as due to alcohol impairment or drug consumption. In some cases this could be done by providing at least some minimum level of real-time or substantially real-time measurement of differential complexity.

For example, impairment could manifest itself though loss of alertness. A diagnosis system might test the driver of a vehicle in real-time or substantially real time. If some impairment is detected, the diagnosis system could then warn the driver or take other suitable action (i.e., disabling the vehicle, notifying authorities, etc.).

Similar to a “black box” flight recorder, a diagnosis system could be used as a recorder in a vehicle to record the cerebral activity during a trip and give indications of alertness levels, and potentially warn the driver that it is not safe to operate the vehicle. In some cases, these warnings could be logged.

In general, some of the teachings herein may be useful toward aiding in implementing strategies for reducing economic, social, health and safety issues related to disturbed sleep. Public policy has helped reduce the risk of automobile crash fatalities mediated by use of alcohol. Similarly, sleepiness can be a serious risk factor and policies and technological means should be developed to monitor and restrict sleepy drivers from operating automobiles.

According to the teachings herein, a new method for hypnogram generation based on physical principles may be possible. Using conventional approaches to sleep medicine, it was not possible to perform sleep diagnosis in a patient's home. However, the systems and methods described herein may allow for the investigation of cerebral aspects of sleep and open the door to full unattended PSG testing in the patient's home.

Sleep is a very important aspect of our lives and healthy sleep an important component in the general health of individuals. At present, sleep health is mostly ignored by the family practice, and it is imperative that this changes.

Some of the systems described herein may permit the implementation of unattended sleep testing, initiated by family practice, in the patient's home, without the need for sleep laboratories. This is useful due to the large incidence of sleep related problems that pass undiagnosed because a significant fraction of the population doesn't go through sleep laboratories.

In general, the family medical practice should be the front line of defense in the detection of sleep related problems. In most medical specialties the patient reaches the specialist only after a referral from the family physician has been made. On the one hand, the family physician is not conventionally equipped for primary sleep diagnostics and a large group of patients pass untreated with numerous long term health consequences (development of cardiac problems, Alzheimer's disease, etc.). The systems and methods described herein have the potential to bring about a paradigm shift in primary diagnostics with large implications for the general health of the population.

For instance, the systems described herein may permit sleep laboratories to cover a larger number of patients at a significantly reduced cost. This can be done competently with comparable information as could be obtained using a “fast track” study (i.e. without any specific information to suggest for example that a full EEG montage is required). Standard sleep laboratory use could then become a resource for complex and unusual patients/circumstances, while most testing of patients will be done in their homes.

Patients often come to a sleep clinic with a complaint of sleepiness and/or fatigue. Some of the systems as described herein will equate to an in-laboratory process (e.g. REM vs. NREM apnea rates), and may also allow better assessment of insomnia which in general has not been subject to PSG study because of the perception of the cost-benefit ratio not being “value for money”. This may open the door to better diagnosis (including misdiagnosis of depression) and long term tracking of function.

In yet another embodiment, the teachings herein may permits pre-surgical screening of patients for the prediction of potential problems during and after anesthesia. It is a known fact that there is a close relationship between sleep and anesthesia. Clinical studies have shown that patients experiencing respiratory problems during sleep are at risk for developing complications during and after administering various anesthetic regimens. There are indications that pre-surgical screening of respiratory problems during sleep will become the standard of care in the near future due to significant morbidity and mortality rates attached to problems during and after anaesthesia. Currently the only solution that takes into consideration the cerebral aspect of respiration is possible through costly tests available in sleep laboratories. In addition there is cost to the patient due to travel and possible days away from work. Sleep laboratories would not be able to test the large volumes of patients that undergo surgery.

The systems herein may provide for automated sleep diagnostics for the family practice. A GP can do sleep studies without in depth knowledge about sleep (same applies for other specialties with interest in sleep diagnostics e.g. respirology or psychiatry). The system could then generate a report similar to a blood cell count in hematology, including clinical sleep parameters and if these are out of range he/she can refer the patient to a sleep specialist.

The systems herein may be useful for detecting impairment due to sleepiness, warning and logging risk levels, potentially used for drivers, operators of installations that require increased vigilance and where errors can have catastrophic consequences.

In another, the teachings herein may be useful for due to the observation that increased sleep arousal measured for 10 days per year predicts Alzheimer's disease. This system may offer a low cost alternative to imaging diagnostics, thus facilitating screening tests.

1. A system for determining sleep staging, comprising:
a complexity module operable to measure the complexity of regularities in an EEG channel; and
a stager operable to output at least one corresponding sleep stage.

2. The system of any preceding claim, further comprising another module operable to monitor a non-EEG channel for improving accuracy sleep staging determination.

3. The system of any preceding claim, further comprising at least one pre-processor for filtering at least one channel.

4. The system of any preceding claim, further comprising at least one DPA module operable to provide a rolling distribution of waves in at least one frequency band.

5. The system of any preceding claim, further comprising an EMG analyzer operable to evaluate skeletal EMG.

6. The system of any preceding claim, further comprising a spectrum analyzer operable to detect artifacts and short-lived transients in the EEG channel.

7. The system of any preceding claim, further comprising a REM/SEM detector

8. The system of any preceding claim, wherein the stager further comprises an estimate end-points module.

9. The system of any preceding claim, wherein the stager further comprises an interpreter module operable to EMG activity and outputs representative levels of skeletal muscle tone for wake (W), non REM (NREM) and REM sleep.

10. The system of any preceding claim, wherein the stager further comprises a REM complexity module operable to estimate the complexity of REM sleep.

11. The system of any preceding claim, wherein the stager further comprises a REM boundaries module.

12. The system of any preceding claim, wherein the stager further comprises a synthesize ideal REM module.

13. The system of any preceding claim, wherein the stager further comprises a staging loop module that proceeds epoch-by-epoch and outputs the corresponding sleep stage.

14. The system of any preceding claim, further comprising plurality of electrodes placed on a patients head in a pattern without scalp electrodes.

15. A method for determining sleep stages and generating a hypnogram comprising measuring a complexity of an EEG channel.

16. Systems and methods for diagnosis of sleep, comprising:
a specific electrode configuration having at least one of A1-REF and A2-REF or another ear application;
wherein the electrode configuration is used for at least one of:
generating a hypnogram;
determining the state of consciousness of a patient;
or for any other application.

17. Systems and methods for detecting impairment due to sleepiness, including at least one of:
monitoring a subject;
determining when the subject is experiencing a sleep state associated with impairment;
warning the subject of the impairment;
logging at least one risk level associated with the impairment.

18. The systems and methods of claim 17 used for at least one of:
drivers of vehicles;
operators of installations that require increased vigilance; and
where errors due to impairment of the subject can have negative consequences.

19. Systems and methods for predicting the presence of Alzheimer's disease in a subject, comprising:
monitoring the subject; and
determining that the subject may have Alzheimer's when increased sleep arousal is observed above a particular threshold.

20. The systems and methods of claim 19, wherein the particular threshold is ten days of increased sleep arousal per year.

21. Systems and methods pre-surgical screening of a subject for the prediction of potential problems during and/or after anesthesia, comprising:
monitoring the subject; and
determining that a potential problem is likely based on a diagnosed sleep staging.

22. The use of one or more of the systems as claimed in claims 1 to 14 to diagnose sleep.

23. A system or method for diagnosis sleep including one or more of the elements or steps all as generally and specifically described herein
